(19) United States
(12) Patent Application Publication
(10) Pub. No.: US 2025/0359875 A1
(43) Pub. Date: Nov. 27, 2025

(54) GUIDES, INSTRUMENTATION, AND METHODS OF USE AND ASSEMBLY FOR OSTEOTOMY PROCEDURES

(71) Applicant: Paragon 28, Inc., Englewood, CO (US)

(72) Inventors: Joseph DOGUÉ, Aurora, CO (US); Adam JENSEN, Golden, CO (US); Madeline Kay SHANAHAN, Lakewood, CO (US)

(73) Assignee: Paragon 28, Inc., Englewood, CO (US)

(21) Appl. No.: 19/289,698

(22) Filed: Aug. 4, 2025

Related U.S. Application Data
(63) Continuation of application No. PCT/US2024/014478, filed on Feb. 5, 2024.
(60) Provisional application No. 63/483,265, filed on Feb. 3, 2023.

(51) Int. Cl. A61B 17/17

(52) U.S. Cl. CPC A61B 17/1775

(57) ABSTRACT

Instruments, devices, systems and methods for maintaining, correcting and/or fusing joint deformities are disclosed. Guide systems including an alignment block and a cut guide removably coupled to the alignment block. Osteotomy guides including an alignment block and a cut guide removably coupled to the alignment block. Osteotomy guides including a body, a first alignment member coupled to and extending away from a superior surface of the body, and a second alignment member coupled to and extending away from an inferior surface of the body. Methods of assembling and using the osteotomy guides and systems for maintaining, correcting and/or fusing joint deformities are also disclosed.

### CROSS REFERENCE TO RELATED APPLICATIONS

This application is a continuation of PCT Application No. PCT/US2024/014478 filed Feb. 5, 2024, and entitled “Guides, Instrumentation, and Methods of Use and Assembly for Osteotomy Procedures,” which claims priority benefit under 35 U.S.C. § 119(e) to U.S. provisional application No. 63/483,265, filed on Feb. 3, 2023 and entitled Guides, Instrumentation, and Methods of Use and Assembly for Osteotomy Procedures, which are incorporated herein by reference in their entireties.

### TECHNICAL FIELD

The present disclosure relates generally to general, podiatric, and orthopaedic surgery related to joint deformities and injuries. More specifically, but not exclusively, the present disclosure relates to guides, devices, instruments, systems and methods for maintaining, correcting and/or resurfacing bones.

### BACKGROUND OF THE INVENTION

Supramalleolar osteotomies (SMOs) are commonly performed to the distal tibia in order to correct varus and valgus ankles. In some cases, this procedure may be performed as a joint-reserving procedure in order to address arthritis resulting from the asymmetry of varus and valgus ankles. In this procedure, the spatial relationship between the talus and the tibia is manipulated and realigned in order to normalize loading of the ankle joint.

SMOs are typically either opening or closing osteotomy procedures. In an opening SMO, a wedge, for example, a graft that may be trimmed to a desired size, is placed in the distal tibia and secured by hardware, for example, a plate. In a closing SMO, a portion of the distal tibia is removed with the void then closed and the tibia similarly secured with hardware.

Additionally, SMOs may be performed on different surfaces of the distal tibia, for example, medial SMOs and anterior SMOs. Further SMOs may be performed using patient specific instrumentation (PSI) which may include various components created to accommodate an anatomy of a patient and guide one or more cuts, placement of pins and/or k-wires, etc.

Typically, an osteotomy procedure is performed with off-the-shelf plating (often universal) and grafts, and any guides incorporated in the procedure are not PSI. It is common for anterior procedures to include a physician selecting a straight plate, pinning a distal portion of the plate to the anterior tibia, selecting a hole of the plate proximal relative to the pins, and drilling an arc along a path that overlaps said hole. Accordingly, various aspects of the procedure are not as precise as when using PSI. For anterior osteotomies, grafts are not typically used. However, grafts are commonly used in medial osteotomy procedures.

Thus, it is an object of the present disclosure to overcome one or more of the above-described drawbacks and/or disadvantages of the currently available systems.

### SUMMARY OF THE INVENTION

The present disclosure is directed toward guides, devices, instruments, systems and methods for maintaining, correcting and/or resurfacing bones.

In one aspect of the present disclosure provided herein, is a guide system. The system including an alignment block and-a cut guide removably coupled to the alignment block.

In another aspect of the present disclosure provided herein is an osteotomy guide. The osteotomy guide including an alignment block and a cut guide removably coupled to the alignment block.

In yet another aspect of the present disclosure provided herein, is an osteotomy guide. The osteotomy guide including a body, a first alignment member coupled to and extending away from a superior surface of the body, and a second alignment member coupled to and extending away from an inferior surface of the body.

Finally, the present disclosure also provides herein methods for assembling and using osteotomy guides and osteotomy guide systems.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 is a first perspective view of an osteotomy guide, in accordance with an aspect of the present disclosure;

FIG. 2 is a second perspective view of the osteotomy guide of FIG. 1, in accordance with an aspect of the present disclosure;

FIG. 3 is a first side view of the osteotomy guide of FIG. 1, in accordance with an aspect of the present disclosure;

FIG. 4 is a second side view of the osteotomy guide of FIG. 1, in accordance with an aspect of the present disclosure;

FIG. 5 is a first end view of the osteotomy guide of FIG. 1, in accordance with an aspect of the present disclosure;

FIG. 6 is a second end view of the osteotomy guide of FIG. 1, in accordance with an aspect of the present disclosure;

FIG. 7 is a superior view of the osteotomy guide of FIG. 1, in accordance with an aspect of the present disclosure;

FIG. 8 is an inferior view of the osteotomy guide of FIG. 1, in accordance with an aspect of the present disclosure;

FIG. 9 is an exploded, first perspective view of the osteotomy guide of FIG. 1, in accordance with an aspect of the present disclosure;

FIG. 10 is an exploded, second perspective view of the osteotomy guide of FIG. 1, in accordance with an aspect of the present disclosure;

FIG. 11 is an exploded, first side view of the osteotomy guide of FIG. 1, in accordance with an aspect of the present disclosure;

FIG. 12 is an exploded, second side view of the osteotomy guide of FIG. 1, in accordance with an aspect of the present disclosure;

FIG. 13 is an exploded, first end view of the osteotomy guide of FIG. 1, in accordance with an aspect of the present disclosure;

FIG. 14 is an exploded, second end view of the osteotomy guide of FIG. 1, in accordance with an aspect of the present disclosure;

FIG. 15 is an exploded, superior view of the osteotomy guide of FIG. 1, in accordance with an aspect of the present disclosure;

FIG. 16 is an exploded, inferior view of the osteotomy guide of FIG. 1, in accordance with an aspect of the present disclosure;

FIG. 17 depicts one embodiment of a surgical method for using the osteotomy guide of FIG. 1, in accordance with an aspect of the present disclosure;

FIG. 18 is a first perspective view of another embodiment of an osteotomy guide positioned on a patient's bone, in accordance with an aspect of the present disclosure;

FIG. 19 is a front view of the osteotomy guide of FIG. 18 positioned on a patient's bone, in accordance with an aspect of the present disclosure;

FIG. 20 is a first perspective view of the osteotomy guide of FIG. 18, in accordance with an aspect of the present disclosure;

FIG. 21 is a second perspective view of the osteotomy guide of FIG. 18, in accordance with an aspect of the present disclosure;

FIG. 22 is a first side view of the osteotomy guide of FIG. 18, in accordance with an aspect of the present disclosure;

FIG. 23 is a second side view of the osteotomy guide of FIG. 18, in accordance with an aspect of the present disclosure;

FIG. 24 is a first end view of the osteotomy guide of FIG. 18, in accordance with an aspect of the present disclosure;

FIG. 25 is a second end view of the osteotomy guide of FIG. 18, in accordance with an aspect of the present disclosure;

FIG. 26 is a superior view of the osteotomy guide of FIG. 18, in accordance with an aspect of the present disclosure;

FIG. 27 is an inferior view of the osteotomy guide of FIG. 18, in accordance with an aspect of the present disclosure;

FIG. 28 is an exploded, first perspective view of the osteotomy guide of FIG. 18, in accordance with an aspect of the present disclosure;

FIG. 29 is an exploded, second perspective view of the osteotomy guide of FIG. 18, in accordance with an aspect of the present disclosure;

FIG. 30 is an exploded, first side view of the osteotomy guide of FIG. 18, in accordance with an aspect of the present disclosure;

FIG. 31 is an exploded, second side view of the osteotomy guide of FIG. 18, in accordance with an aspect of the present disclosure;

FIG. 32 is an exploded, first end view of the osteotomy guide of FIG. 18, in accordance with an aspect of the present disclosure;

FIG. 33 is an exploded, second end view of the osteotomy guide of FIG. 18, in accordance with an aspect of the present disclosure;

FIG. 34 is an exploded, superior view of the osteotomy guide of FIG. 18, in accordance with an aspect of the present disclosure;

FIG. 35 is an exploded, inferior view of the osteotomy guide of FIG. 18, in accordance with an aspect of the present disclosure;

FIGS. 36-51 depict a surgical method for using the osteotomy guide of FIG. 18, in accordance with an aspect of the present disclosure;

FIG. 52 is a first perspective view of another osteotomy guide positioned on a patient's bone, in accordance with an aspect of the present disclosure;

FIG. 53 is a second perspective view of the osteotomy guide of FIG. 52 positioned on a patient's bone, in accordance with an aspect of the present disclosure;

FIG. 54 is a first perspective view of the osteotomy guide of FIG. 52, in accordance with an aspect of the present disclosure;

FIG. 55 is a second perspective view of the osteotomy guide of FIG. 52, in accordance with an aspect of the present disclosure;

FIG. 56 is a first side view of the osteotomy guide of FIG. 52, in accordance with an aspect of the present disclosure;

FIG. 57 is a second side view of the osteotomy guide of FIG. 52, in accordance with an aspect of the present disclosure;

FIG. 58 is a fist end view of the osteotomy guide of FIG. 52, in accordance with an aspect of the present disclosure;

FIG. 59 is a second view of the osteotomy guide of FIG. 52, in accordance with an aspect of the present disclosure;

FIG. 60 is a superior view of the osteotomy guide of FIG. 52, in accordance with an aspect of the present disclosure;

FIG. 61 is an inferior view of the osteotomy guide of FIG. 52, in accordance with an aspect of the present disclosure;

FIG. 62 is a first perspective view of another osteotomy guide positioned on a patient's bone, in accordance with an aspect of the present disclosure;

FIG. 63 is a second perspective view of the osteotomy guide of FIG. 62 positioned on a patient's bone, in accordance with an aspect of the present disclosure;

FIG. 64 is a first perspective view of the osteotomy guide of FIG. 62, in accordance with an aspect of the present disclosure;

FIG. 65 is a second perspective view of the osteotomy guide of FIG. 62, in accordance with an aspect of the present disclosure;

FIG. 66 is a first side view of the osteotomy guide of FIG. 62, in accordance with an aspect of the present disclosure;

FIG. 67 is a second side view of the osteotomy guide of FIG. 62, in accordance with an aspect of the present disclosure;

FIG. 68 is a first end view of the osteotomy guide of FIG. 62, in accordance with an aspect of the present disclosure;

FIG. 69 is a second end view of the osteotomy guide of FIG. 62, in accordance with an aspect of the present disclosure;

FIG. 70 is a superior view of the osteotomy guide of FIG. 62, in accordance with an aspect of the present disclosure;

FIG. 71 is an inferior view of the osteotomy guide of FIG. 62, in accordance with an aspect of the present disclosure;

FIG. 72 depict a surgical method for using the osteotomy guides of FIGS. 54 and 64, in accordance with an aspect of the present disclosure;

FIG. 73 a first perspective view of another osteotomy guide positioned on a patient's bone, in accordance with an aspect of the present disclosure;

FIG. 74 is a first side view of the osteotomy guide of FIG. 73 positioned on a patient's bone, in accordance with an aspect of the present disclosure;

FIG. 75 is a first perspective view of the osteotomy guide of FIG. 73, in accordance with an aspect of the present disclosure;

FIG. 76 is a second perspective view of the osteotomy guide of FIG. 73, in accordance with an aspect of the present disclosure;

FIG. 77 is a first side view of the osteotomy guide of FIG. 73, in accordance with an aspect of the present disclosure;

FIG. 78 is a second side view of the osteotomy guide of FIG. 73, in accordance with an aspect of the present disclosure;

FIG. 79 is a first end view of the osteotomy guide of FIG. 73, in accordance with an aspect of the present disclosure;

FIG. 80 is a second end view of the osteotomy guide of FIG. 73, in accordance with an aspect of the present disclosure;

FIG. 81 is a superior view of the osteotomy guide of FIG. 73, in accordance with an aspect of the present disclosure;

FIG. 82 is an inferior view of the osteotomy guide of FIG. 73, in accordance with an aspect of the present disclosure;

FIG. 83 depict a surgical method for using the osteotomy guides of FIG. 73, in accordance with an aspect of the present disclosure;

FIG. 84 is a first perspective view of another osteotomy guide system positioned on a patient's bone, in accordance with an aspect of the present disclosure;

FIG. 85 is a lateral view of the osteotomy guide system of FIG. 84 positioned on a patient's bone, in accordance with an aspect of the present disclosure;

FIG. 86 is a first perspective view of the osteotomy guide system of FIG. 84, in accordance with an aspect of the present disclosure;

FIG. 87 is a second perspective view of the osteotomy guide system of FIG. 84, in accordance with an aspect of the present disclosure;

FIG. 88 is a first side view of the osteotomy guide system of FIG. 84, in accordance with an aspect of the present disclosure;

FIG. 89 is a second side view of the osteotomy guide system of FIG. 84, in accordance with an aspect of the present disclosure;

FIG. 90 is a first end view of the osteotomy guide system of FIG. 84, in accordance with an aspect of the present disclosure;

FIG. 91 is a second end view of the osteotomy guide system of FIG. 84, in accordance with an aspect of the present disclosure;

FIG. 92 is a superior view of the osteotomy guide system of FIG. 84, in accordance with an aspect of the present disclosure;

FIG. 93 is an inferior view of the osteotomy guide system of FIG. 84, in accordance with an aspect of the present disclosure;

FIG. 94 is a first perspective view of a fibula cut guide system positioned on a patient's bone, in accordance with an aspect of the present disclosure;

FIG. 95 is a front view of the fibula cut guide system of FIG. 94 positioned on a patient's bone, in accordance with an aspect of the present disclosure;

FIG. 96 is a first perspective view of the fibula cut guide system of FIG. 94, in accordance with an aspect of the present disclosure;

FIG. 97 is a second perspective view of the fibula cut guide system of FIG. 94, in accordance with an aspect of the present disclosure;

FIG. 98 is a first side view of the fibula cut guide system of FIG. 94, in accordance with an aspect of the present disclosure;

FIG. 99 is a second side view of the fibula cut guide system of FIG. 94, in accordance with an aspect of the present disclosure;

FIG. 100 is a first end view of the fibula cut guide system of FIG. 94, in accordance with an aspect of the present disclosure;

FIG. 101 is a second end view of the fibula cut guide system of FIG. 94, in accordance with an aspect of the present disclosure;

FIG. 102 is a superior view of the fibula cut guide system of FIG. 94, in accordance with an aspect of the present disclosure;

FIG. 103 is an inferior view of the fibula cut guide system of FIG. 94, in accordance with an aspect of the present disclosure;

FIG. 104 is a first perspective view of an assembled tibia alignment guide system, in accordance with an aspect of the present disclosure;

FIG. 105 is a second perspective view of the assembled tibia alignment guide system of FIG. 104, in accordance with an aspect of the present disclosure;

FIG. 106 is an exploded, first perspective view of the tibia alignment guide system of FIG. 104, in accordance with an aspect of the present disclosure;

FIG. 107 is an exploded, second perspective view of the tibia alignment guide system of FIG. 104, in accordance with an aspect of the present disclosure;

FIG. 108 is an exploded, first side view of the tibia alignment guide system of FIG. 104, in accordance with an aspect of the present disclosure;

FIG. 109 is an exploded, second side view of the tibia alignment guide system of FIG. 104, in accordance with an aspect of the present disclosure;

FIG. 110 is an exploded, superior view of the tibia alignment guide system of FIG. 104, in accordance with an aspect of the present disclosure;

FIG. 111 is an exploded, inferior view of the tibia alignment guide system of FIG. 104, in accordance with an aspect of the present disclosure;

FIG. 112 is an exploded, first end view of the tibia alignment guide system of FIG. 104, in accordance with an aspect of the present disclosure; and

FIG. 113 is an exploded, second end view of the tibia alignment guide system of FIG. 104, in accordance with an aspect of the present disclosure.

### DETAILED DESCRIPTION FOR CARRYING OUT THE INVENTION

Generally stated, disclosed herein are guides, devices, instruments, systems, and methods for maintaining, correcting and/or resurfacing joint surfaces. Further, methods for using the guides, devices, instruments, systems, and methods for maintaining, correcting and/or resurfacing joint surfaces are discussed.

In this detailed description and the following claims, the words proximal, distal, anterior or plantar, posterior or dorsal, medial, lateral, superior and inferior are defined by their standard usage for indicating a particular part or portion of a bone, instrument, or implant according to the relative disposition of the natural bone or directional terms of reference. For example, “proximal” means the portion of a device or guide nearest the torso, while “distal” indicates the portion of the device or guide farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current devices, systems, instrumentation and methods are described herein with reference to use with the bones of the ankle, the bones of the foot, ankle and lower leg may be used to describe the surfaces, positions, directions or orientations of the devices, systems, instrumentation and methods. Further, the devices, systems, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, systems, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the devices, systems, instrumentation and methods, and the aspects, components, features and the like thereof, described herein with respect to the right leg may be mirrored so that they likewise function with the left leg. Further, the devices, systems, instrumentation and methods, and the aspects, components, features and the like thereof, disclosed herein are described with respect to the leg for brevity purposes, but it should be understood that the devices, systems, instrumentation and methods may be used with other bones of the body having similar structures.

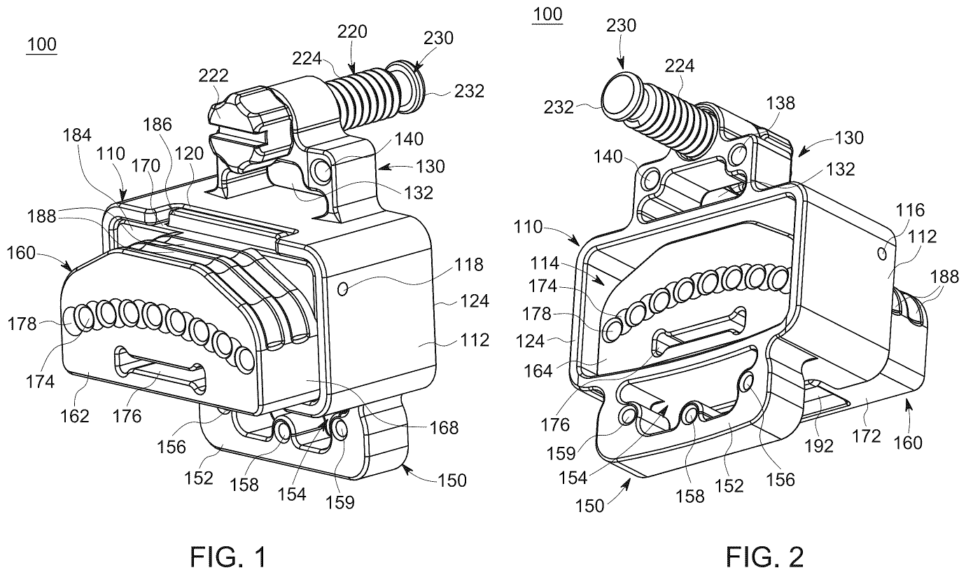

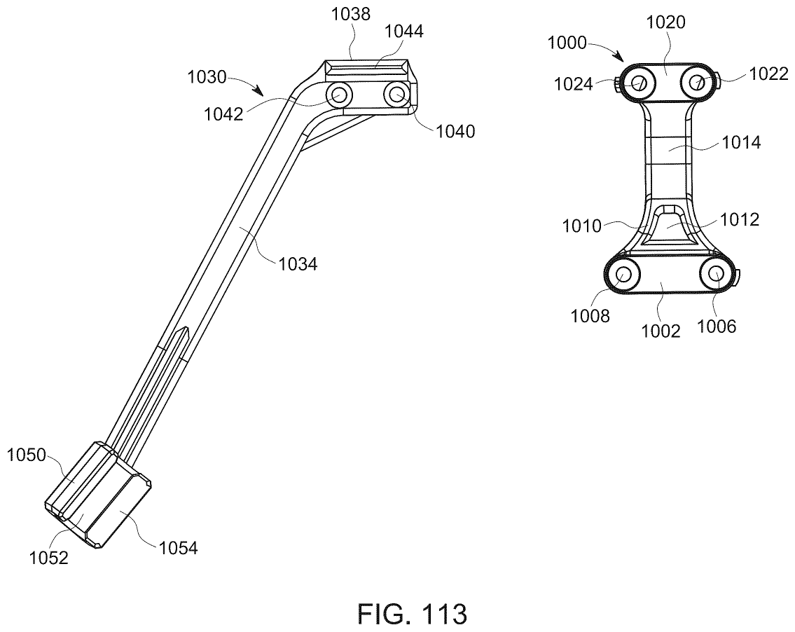

Referring to the drawings, wherein like reference numerals are used to indicate like or analogous components throughout the several views, and with particular reference to FIGS. 1-113, instruments, devices, guides, systems, and methods of using the instruments, devices, guides, and systems for osteotomy procedures are shown.

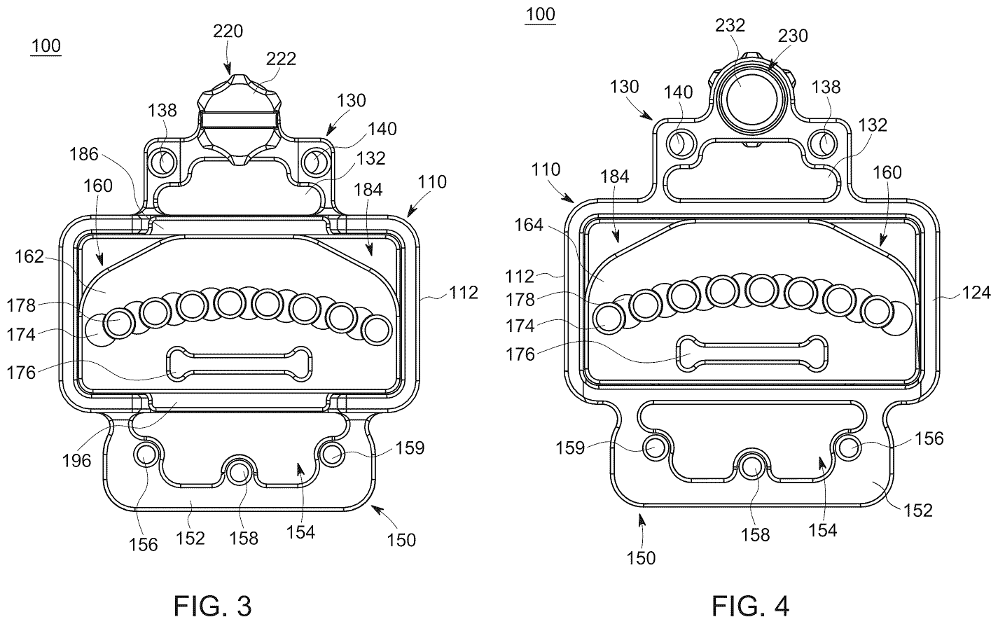

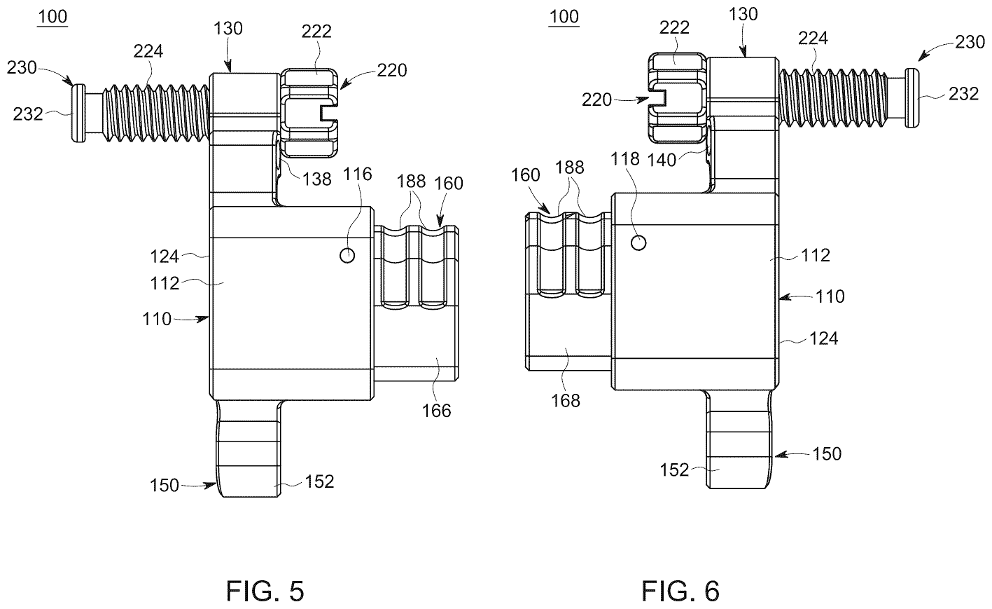

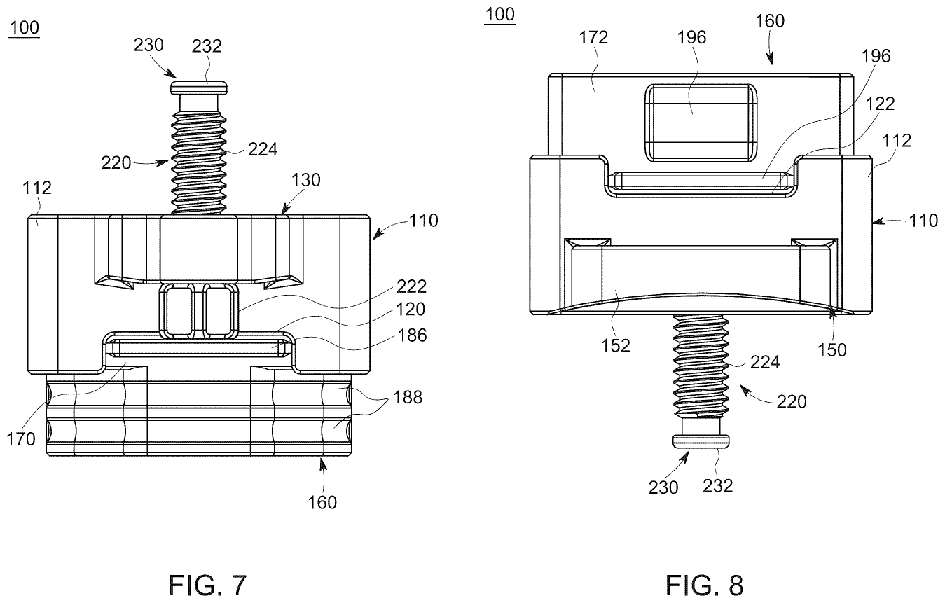

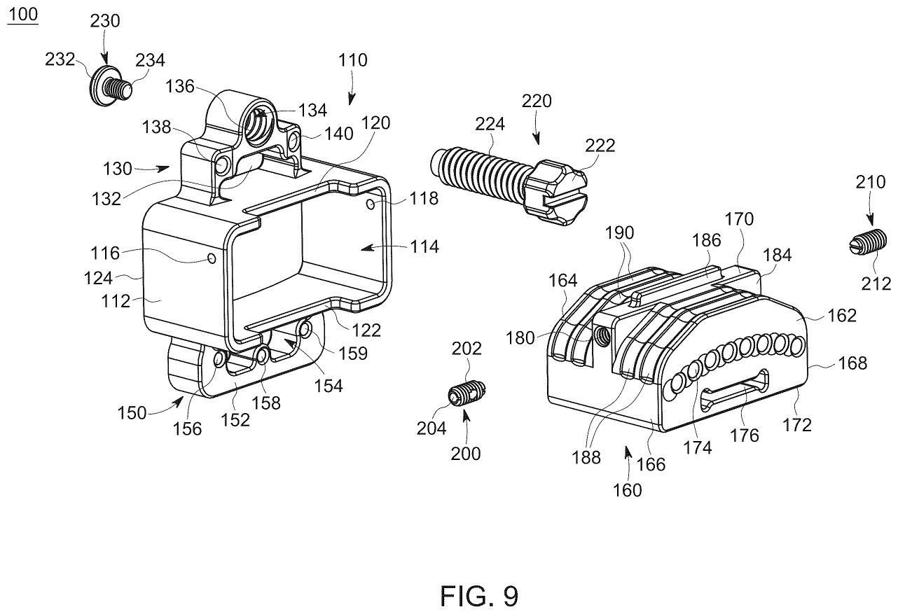

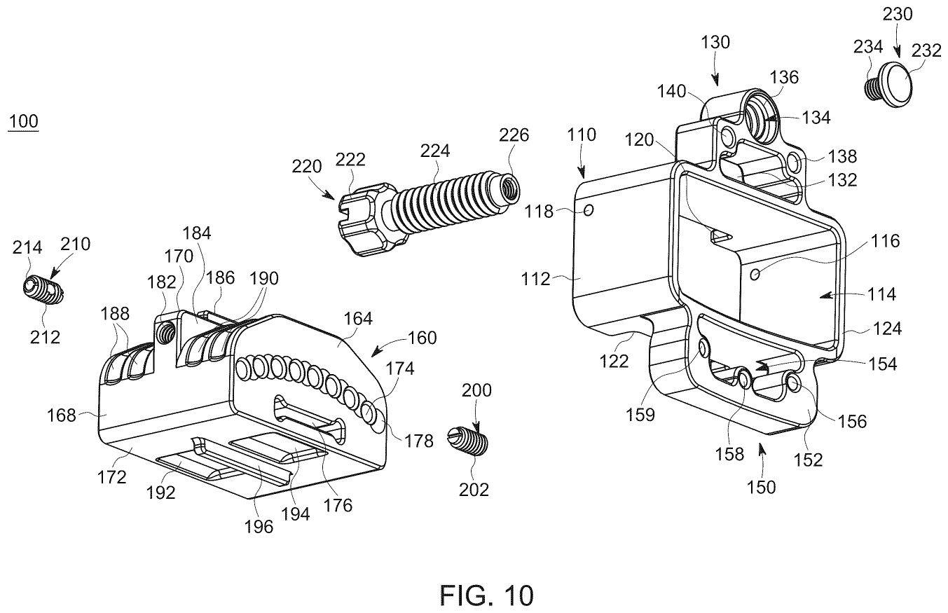

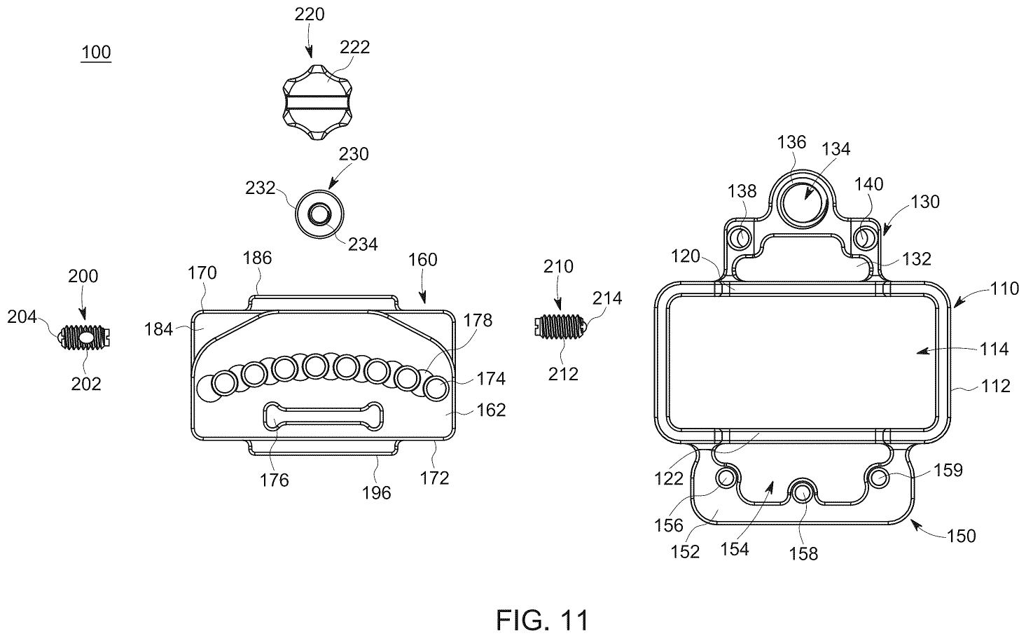

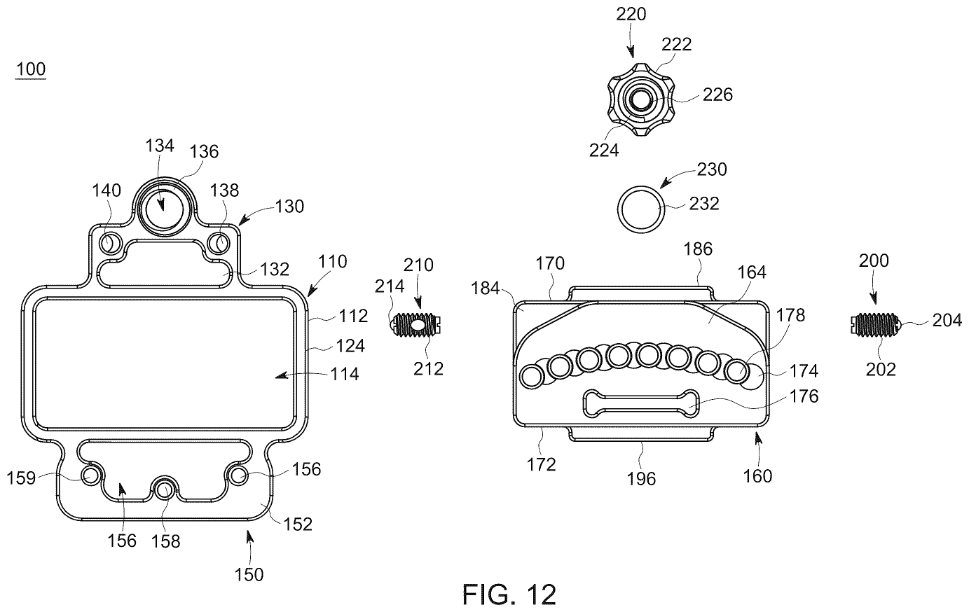

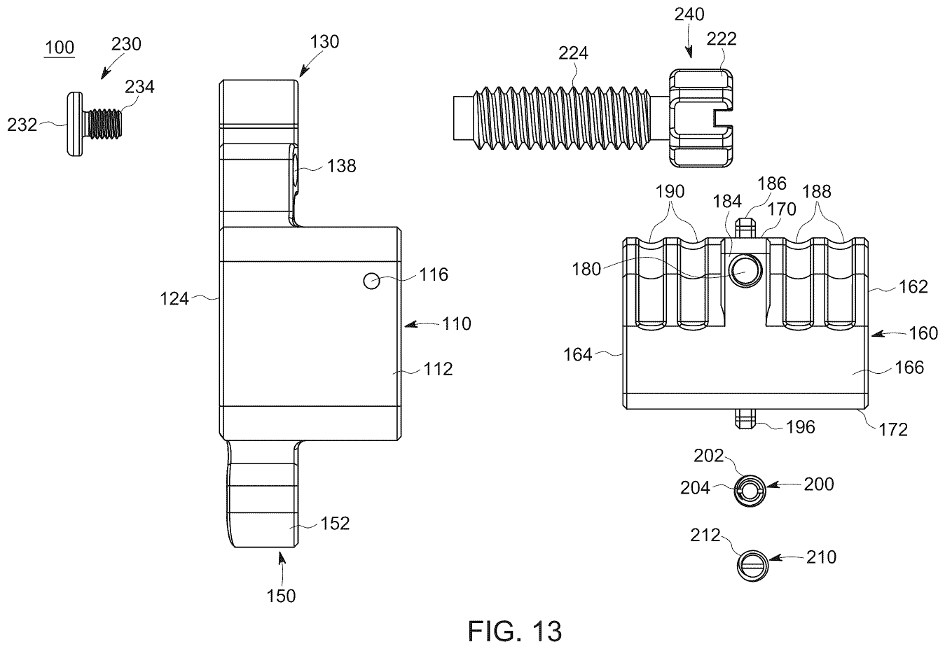

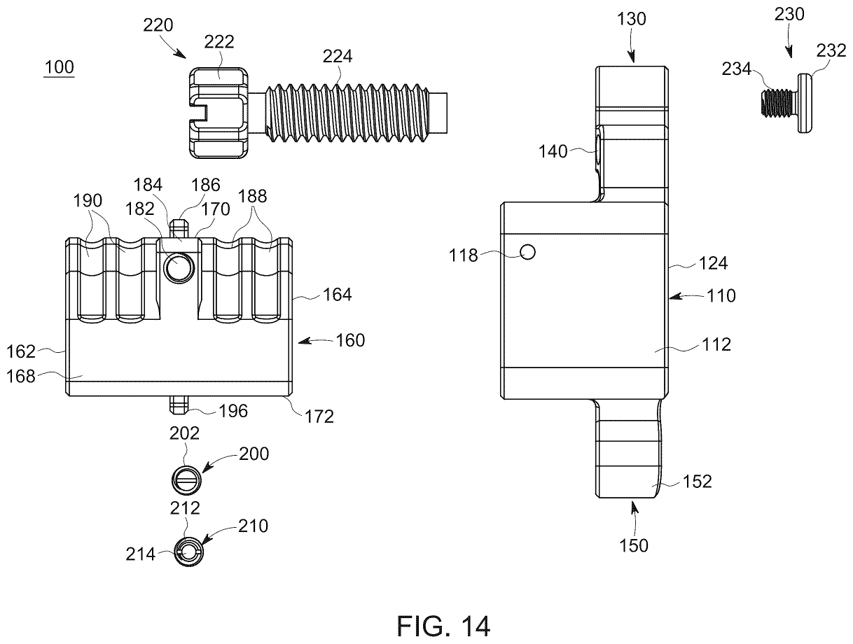

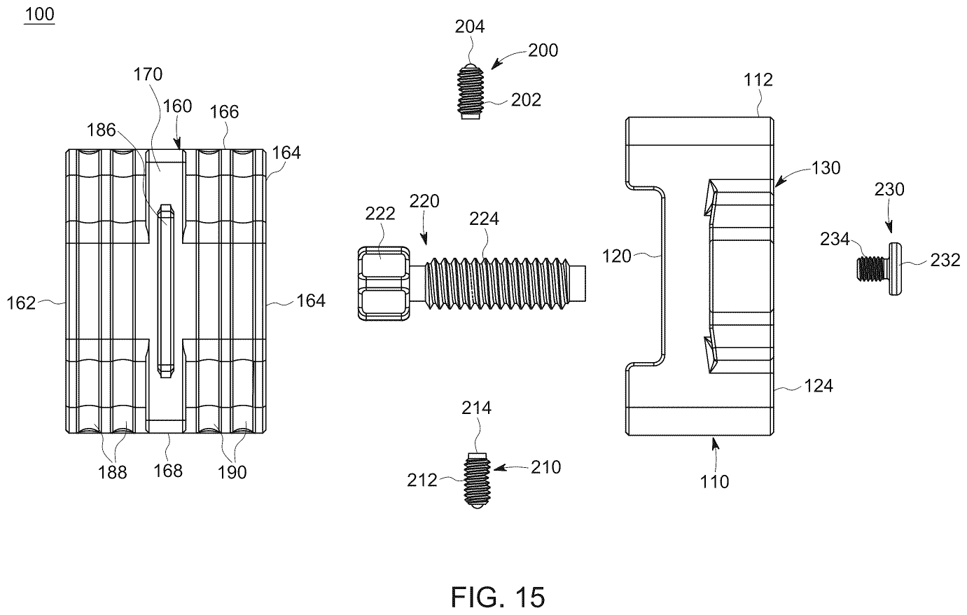

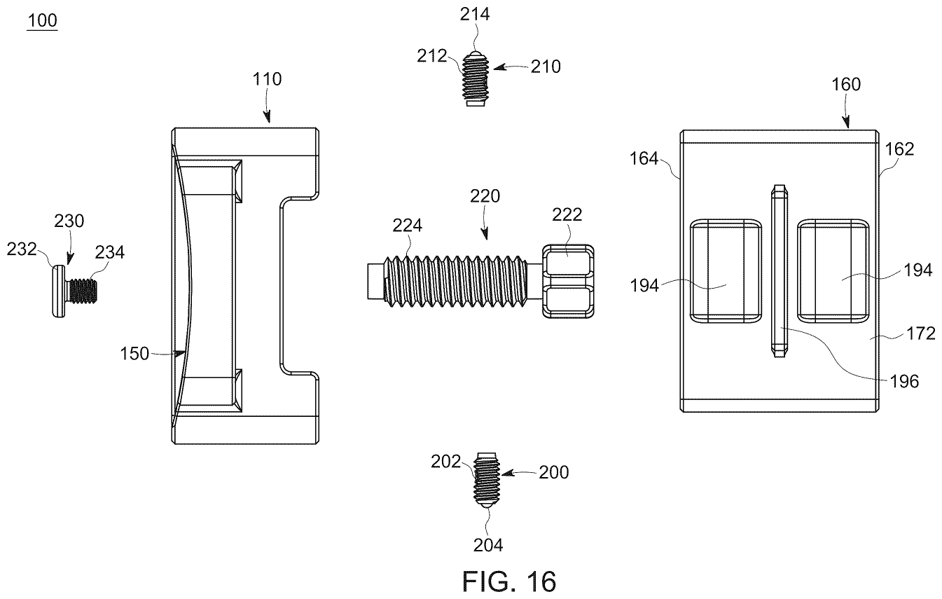

Referring now to FIGS. 1-16, a tibia osteotomy guide or guide system 100 is shown. The osteotomy guide 100 includes an alignment block 110 and a cut guide or cut block 160 removably insertable into the alignment block 110. The osteotomy guide 100 also includes two fasteners or spring plungers 200, 210 for removably coupling the cut guide 160 to the alignment block 110. In addition, the osteotomy guide 100 includes a screw 220 extending through the alignment block 110 and secured using a fastener 230. The osteotomy guide 100 may be used, for example, for anterior osteotomy procedures.

With continued reference to FIGS. 1-16, the alignment block 110 includes a housing 112, a first alignment member 130 extending away from the top surface of the alignment block 110 and a second alignment member 150 extending away from the bottom surface of the alignment block 110. The housing 112 includes a through hole 114 extending through the alignment block 110 from a first side to a second side. The through hole 114 may have, for example, a rectangular shape to correspond to the exterior shape of the cut block 160 or alternatively, may have another shape complementary to the shape of the cut block 160. The housing 112 may include at least one opening 116, 118 extending through the housing 112 from the through hole 114 to an exterior surface of the housing 112. The at least one opening 116, 118 may include a first opening 116 extending through a first end of the housing 112 and a second opening 118 extending through a second end of the housing 112. The first end of the alignment block 110 is positioned opposite the second end of the alignment block 110. In addition, the first opening 116 is positioned opposite the second opening 118, for example, directly opposite. The housing 112 also includes at least one recess 120, 122 extending into a front or anterior surface of the housing 112. The at least one recess 120, 122 may include a first recess 120 positioned at a top of the housing 112 and a second recess 122 positioned at a bottom of the housing 112. The recesses 120, 122 may extend into at least a portion of the housing 112 from a front or anterior side of the osteotomy guide 100. The first recess 120 may be, for example, positioned on the anterior and superior side of the housing 112 opposite the first alignment member 130. The second recess 122 may be, for example, positioned on the anterior and inferior side of the housing 112 opposite the second alignment member 150. The alignment block 110 may also include a bone contacting surface 124 positioned on a back or posterior surface of the alignment block 110. The bone contacting surface 124 may be, for example, shaped to correspond to the shape of the bones on the anterior portion of the ankle bones.

As noted above, the first alignment member 130 may extend away from a top or superior surface of the housing 112. The first alignment member 130 may be coupled to the posterior portion of the superior surface of the housing 112. The first alignment member 130 may include a through hole 132 extending through the member 130 from an anterior side to a posterior side. The through hole 132 may be, for example positioned directly above the superior surface of the housing 112. The through hole 132 may also have, for example, a top hat like shape with a larger opening extending from the superior surface of the housing 112 and a smaller opening positioned directly above the larger opening. In addition, the first alignment member 130 may include a screw hole 134 extending through an upper portion of the first alignment member 130. The screw hole 134 may also extend through the member 130 from an anterior surface to a posterior surface. The screw hole 134 may include threads 136 for receiving a threaded screw, for example, screw 220. The screw hole 134 may be, for example, positioned midway between the first and second or medial and lateral sides of the housing 112. The top surface of the first alignment member 130 surrounding the screw hole 134 may be, for example, rounded or curved. The first alignment member 130 may also include at least one pin hole or alignment opening 138, 140. The at least one pin hole 138, 140 may be, for example, a first pin hole or alignment opening 138 and a second pin hole or alignment opening 140. The openings 138, 140 may be, for example, positioned on either side of the through hole 132 and screw hole 134. The first hole 138 may be, for example, positioned on the first side of the first alignment member 130 and the second hole 140 may be, for example, positioned on a second side of the member 130. The holes 138, 140 may be, for example, converging holes both angled towards the screw hole 134.

As noted above, the second alignment member 150 may extend away from a bottom or inferior surface of the housing 112. The first alignment member 130 and the second alignment member 150 may correspond with upper and lower (e.g., top and bottom, proximal and distal, etc.) edges of a plate which may be implemented in conjunction with the osteotomy guide 100 as components of an osteotomy system. The second alignment member 150 may be coupled to the posterior portion of the inferior surface of the housing 112. The second alignment member 150 may include a through hole 154 extending through the member 150 from an anterior side to a posterior side. The through hole 154 may be, for example positioned directly below the inferior surface of the housing 112. The through hole 154 may also have, for example, a shape with a larger opening extending from the inferior surface of the housing 112 and two smaller openings positioned directly below the larger opening. In addition, the second alignment member 150 may include at least one pin hole or alignment opening 156, 158, 159. The at least one pin hole 156, 158, 159 may be, for example, a first pin hole or alignment opening 156, a second pin hole or alignment opening 158, and a second pin hole or alignment opening 159. The holes 156, 158, 159 may be, for example, positioned below the larger opening of the through hole 154. The first hole 156 may be, for example, positioned near the first side of the second alignment member 150. The second hole 158 may be, for example, positioned between the two smaller openings of the through hole 154. The second hole 158 may be, for example, positioned near a midpoint of the second alignment member 150. The third hole 159 may be, for example, positioned near a second side of the member 150.

The cut guide or cut block 160 includes a first side or front 162 and a second side or back 164 opposite the first side 162. The cut guide 160 also includes a first end 166 and a second end 168 opposite the first end 166. In addition, the cut guide 160 includes a superior side or top 170 and an inferior side or bottom 172 opposite the superior side 170. The cut guide 160 includes a first set of drill or cutting openings 174 extending through the cut guide 160 from the first side 162 to the second side 164. The cut guide 160 also includes a slot or angel wing slot 176 extending through the cut guide 160 from the first side 162 to the second side 164. The slot 176 may have, for example a dog bone shape or the slot 176 may be wider at each end than across the middle. The slot 176 may be configured or sized and shaped to receive instruments, such as, angle wings, lasers, or other alignment instruments for measuring or positioning various components of the osteotomy guide 100. The cut guide 160 also includes a second set of drill or cutting openings 178 extending through the cut guide 160 from the second side 164 to the first side 162. The first set of drill openings 174 will be offset from the second set of drill openings 178. The drill openings 174, 178 may be positioned, for example, in a curve or arc shape as the drill openings 174, 178 extend between the first end 166 and the second end 168. The curved or arc shaped drill openings 174, 178 may have, for example, a concave or convex shape. Alternative drill opening patterns are also contemplated including various drill patterns of various radii. Some alternative drill patterns may not include radii, but instead may include spline curves or other geometric shapes corresponding to a portion of the anatomy of a patient. In addition, alternative cutting blocks 160 are also contemplated including cuts slots (not shown) of various dimensions and/or angles. The cut block 160 may be, for example, reversible allowing for insertion of either the first side 162 or the second side 164 of the cut block 160 into the alignment block 110 for drilling and/or cutting the patient's bone.

The cut guide 160 also includes a first screw hole 180 positioned on and extending into the first end 166 of the cut guide 160. The first screw hole 180 may be positioned near the superior side 170 of the cut guide 160. The first screw hole 180 may also be positioned at or near a midpoint of the cut guide 160 positioned between the first side 162 and the second side 164. The first screw hole 180 may be, for example, threaded. The cut guide 160 may further include a second screw hole 182 positioned on and extending into the second end 168 of the cut guide 160. The second screw hole 182 may be positioned near the superior side 170 of the cut guide 160. The second screw hole 182 may also be positioned at or near a midpoint of the cut guide 160 positioned between the first side 162 and the second side 164. The second screw hole 182 may be, for example, threaded. The screw holes 180, 182 may be, for example, configured or sized and shaped to receive the fasteners 200, 210, respectively.

The top side 170 of the cut guide 160 may include, for example, a superior portion and an inferior portion that is shaped angularly such that the center portion is flat or planar and as the top side 170 extends to each of the first end 166 and second end 168 the top side 170 includes angles toward each of the sides 166, 168. In addition, the top side 170 includes a central portion 184 giving the cut guide 160, for example, a rectangular shape near a midpoint between the first side 162 and second side 164 of the cut guide 160. The screw holes 180, 182 extend into the central portion 184. The cut guide 160 also includes a first protrusion 186 extending away from a portion of a top surface of the central portion 184. The first protrusion 186 has a length shorter than the length of the cut guide 160 extending between the first end 166 and second end 168. The top side 170 may also include at least one first groove 188 extending into a portion of the top side 170 of the cut guide 160 between the central portion 184 and the first side 162 of the cut guide 160. As shown, the at least one first groove 188 may be, for example, two grooves 188, although alternative numbers of grooves 188 are also contemplated. The top side 170 may also include at least one second groove 190 extending into a portion of the top side 170 of the cut guide 160 between the central portion 184 and the second side 164 of the cut guide 160. As shown, the at least one second groove 190 may be, for example, two grooves 190, although alternative numbers of grooves 190 are also contemplated.

The bottom side 172 of the cut guide 160 may include a first recess 192 and a second recess 194 each extending into the bottom side 172. The bottom side 172 may also include a second protrusion 196 extending away from a portion of the bottom side 172 of the cut guide 160. The second protrusion 196 may be, for example, positioned at a midpoint between the first side 162 and the second side 164 of the cut guide 160. The second protrusion 196 may have a length shorter than the length of the cut guide 160 extending between the first end 166 and second end 168. The first recess 192 may be positioned between the second protrusion 196 and the first side 162. The first recess 192 may have, for example, a length shorter than the length of both the second protrusion 196 and the cut guide 160. The second recess 194 may be positioned between the second protrusion 196 and the second side 164. The second recess 194 may have, for example, a length shorter than the length of both the second protrusion 196 and the cut guide 160. The first recess 192 and second recess 194 may be, for example, identically sized and shaped. The first protrusion 186 and the second protrusion 196 may have, for example, the same size and shape. The first protrusion 186 and the second protrusion 196 may be, for example, configured or sized and shaped to be received within the first recess 120 and second recess 122, respectively, of the alignment block 110. The protrusions 186, 196 engaging the recesses 120, 122 allows for the cut block 160 to be correctly placed within the alignment block 110 for drilling and/or cutting.

The osteotomy guide 100 also includes two spring plungers or fasteners 200, 210. The first spring plunger or fastener 200 includes threads 202 positioned around the exterior of the fastener 200. In addition the first fastener 200 includes an engagement member 204 on a first end of the fastener 200. The first fastener 200 may be, for example, threadingly inserted into the first screw hole 180 of the cut block 160 and the engagement member 204 is configured or sized and shaped to engage the first opening 116 in the alignment block 110 upon insertion of the cut block 160 into the alignment block 110. The second spring plunger or fastener 210 includes threads 212 positioned around the exterior of the fastener 210. In addition the second fastener 210 includes an engagement member 214 on a second end of the fastener 210. The second fastener 210 may be, for example, threadingly inserted into the second screw hole 182 of the cut block 160 and the engagement member 214 is configured or sized and shaped to engage the second opening 118 in the alignment block 110 upon insertion of the cut block 160 into the alignment block 110.

Finally, the osteotomy guide 100 includes a set screw or screw 220 and a fastener 230. The screw 220 includes a head 222 and a shaft 224 extending from a second side of the head 222. The shaft 224 may be, for example, threaded along a portion or the entire length of the shaft 224. The screw 220 may also include an opening 226 extending into the shaft 224 from the end opposite the head 222. The opening 226 may be, for example, threaded to receive or engage the fastener 230. The threads of the shaft 224 may engage the screw hole 134 of the alignment block 110 to allow for adjustment of the contact of the screw 220 with the patient's tibia for positioning of the osteotomy guide 100. The fastener 230 may include a head 232 and a shaft 234 extending from a first side of the head 232. The shaft 234 may be, for example, threaded to engage the threads of the opening 226 of the screw 220. The head 232 may have a flat or planar second side for contacting the bone surface. In addition, the diameter of the head 232 may be, for example, the same size as the diameter of the shaft 224.

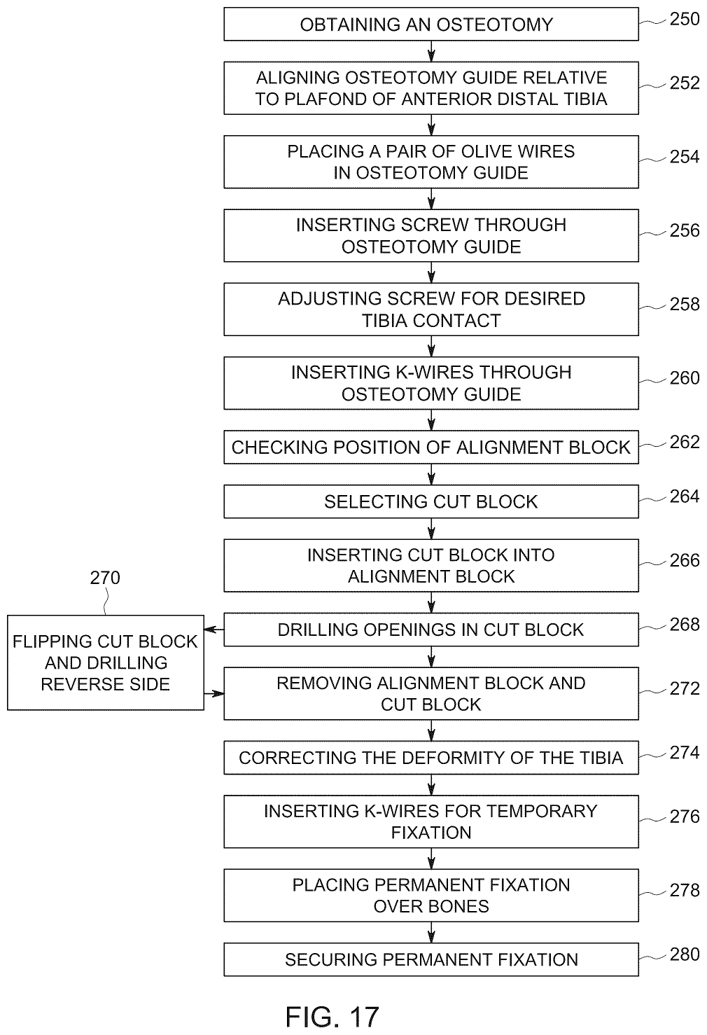

Referring now to FIG. 17, a method for using the osteotomy guide 100 is shown. The method includes obtaining an osteotomy guide 250. The method also includes aligning a bottom portion of the osteotomy guide at or relative to the plafond of the anterior distal tibia 252. Next, the method includes placing a pair of olive wires within a pair of pin holes or alignment openings in the second alignment member of the osteotomy guide 254. The method further includes inserting a screw through a first alignment member of the osteotomy guide 256 and adjusting the screw for proper tibial contact 258. Adjusting the screw includes spacing the osteotomy guide from the anterior surface of the tibia in the sagittal plane. In addition, the method includes inserting k-wires within the pin holes in the first alignment member of the osteotomy guide 260. The pin holes may, for example, be converging pin holes. The method then includes checking position of alignment block 262 and selecting cut block or cut guide 264. The cut block may be selected by a physician based on radius of the pattern, length of the pattern, for example, arc length, and/or orientation of the pattern, for example, an arc that opens either distally or proximally. The method also includes inserting cut block into alignment block 266 and drilling or cutting through openings in cut block 268. Optionally, the method may include flipping the cut block and drilling through reversed openings 270. After all of the openings have been drilled or cut, the method includes removing the cut block and the alignment block from bones 272. If needed, the method may also include using an instrument, for example, an osteotome or the like, to connect or clean up along the drill pattern to create an elongated slot along the geometry of the holes. Although the cut block should be removed before connecting or cleaning up the drilled or cut openings, the alignment block may be removed before or after connecting or cleaning up the drilled or cut holes in the tibia. Next, the method includes correcting the deformity by adjusting the alignment of the portions of the tibia 274. The adjustment of the tibia may be made, for example, primarily in the coronal plane. In addition, the method includes inserting k-wires for temporary fixation 276. Following temporary fixation, the method includes placing a permanent fixation over the bones of the osteotomy 278. The permanent fixation may be, for example, a plate. Finally, the method includes securing the permanent fixation or plate to the bones 280.

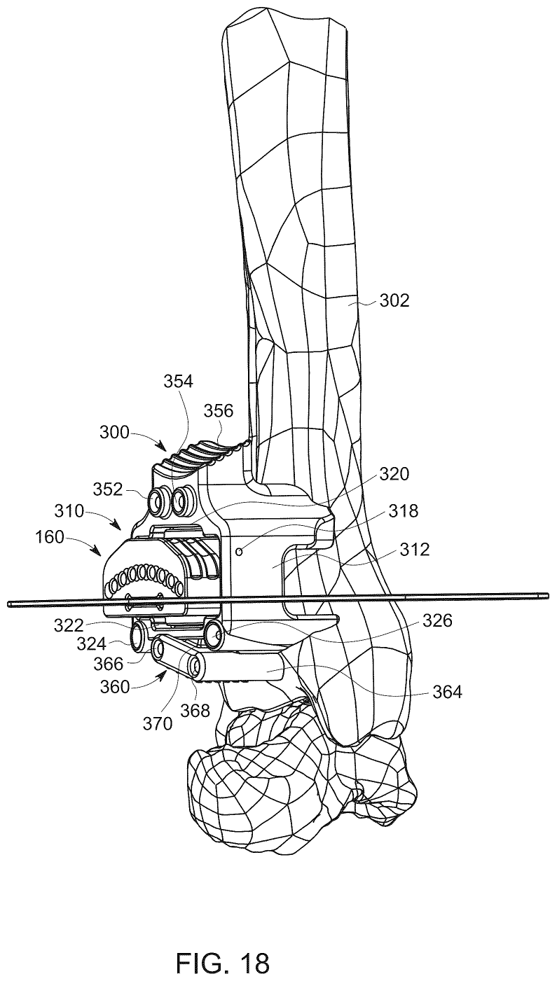

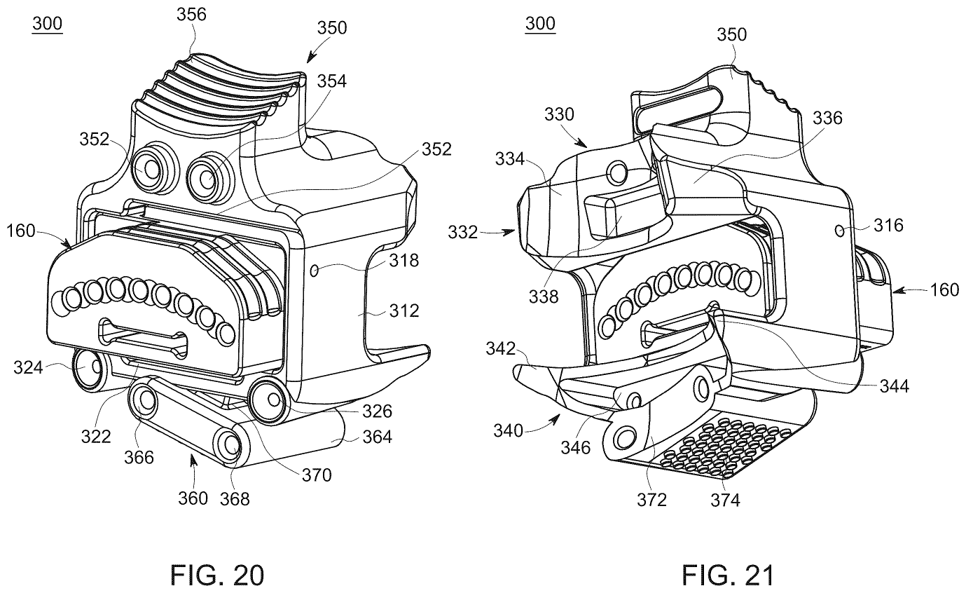

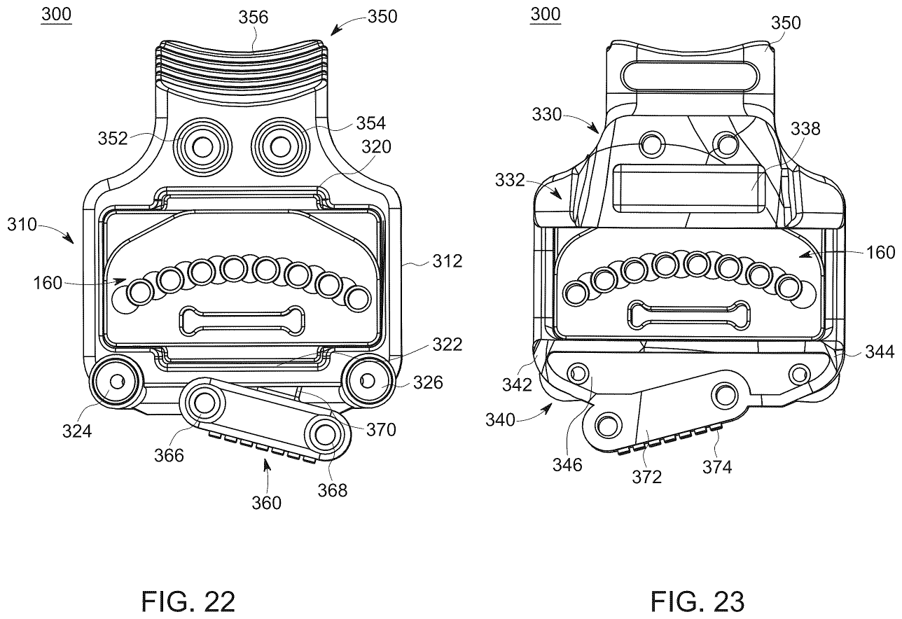

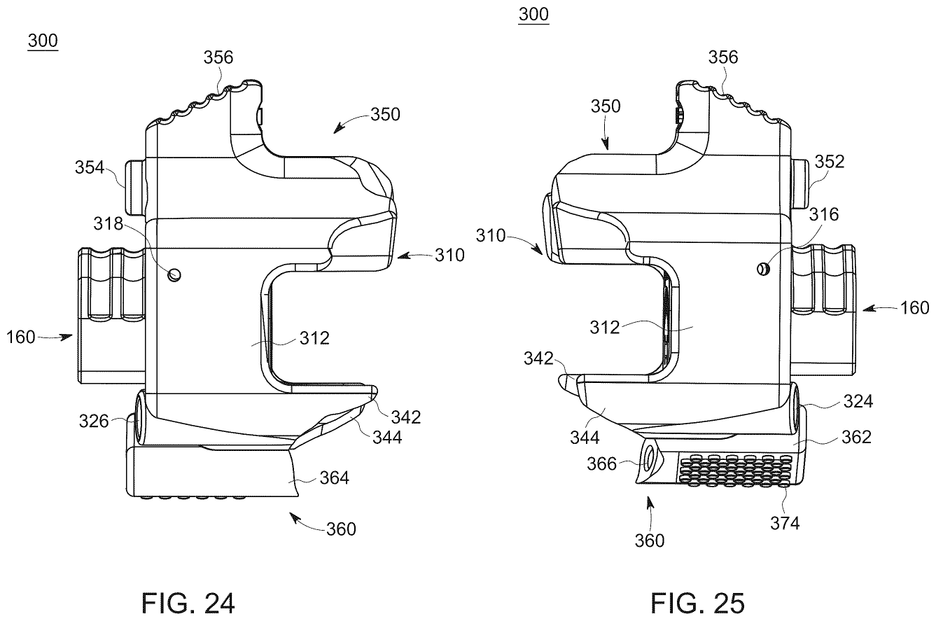

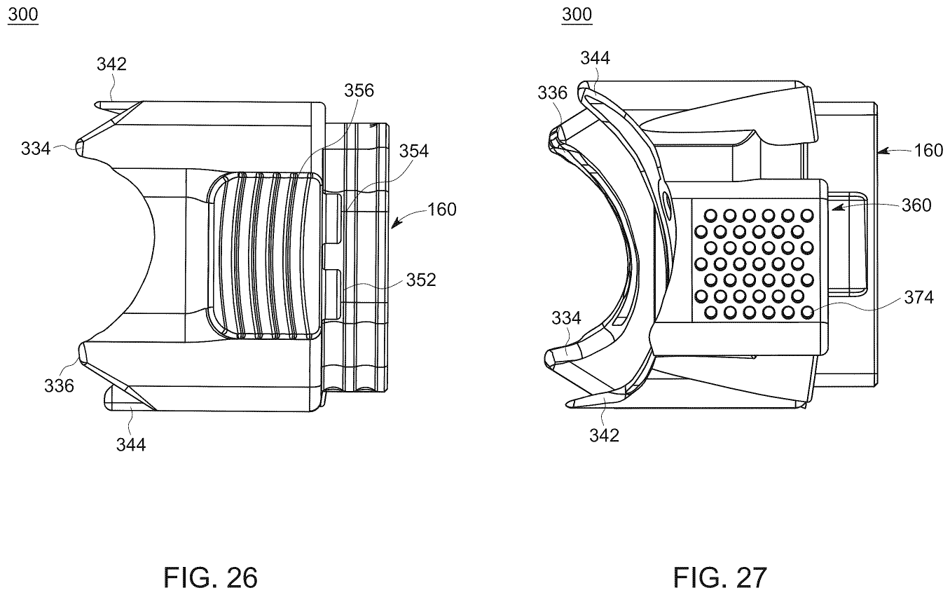

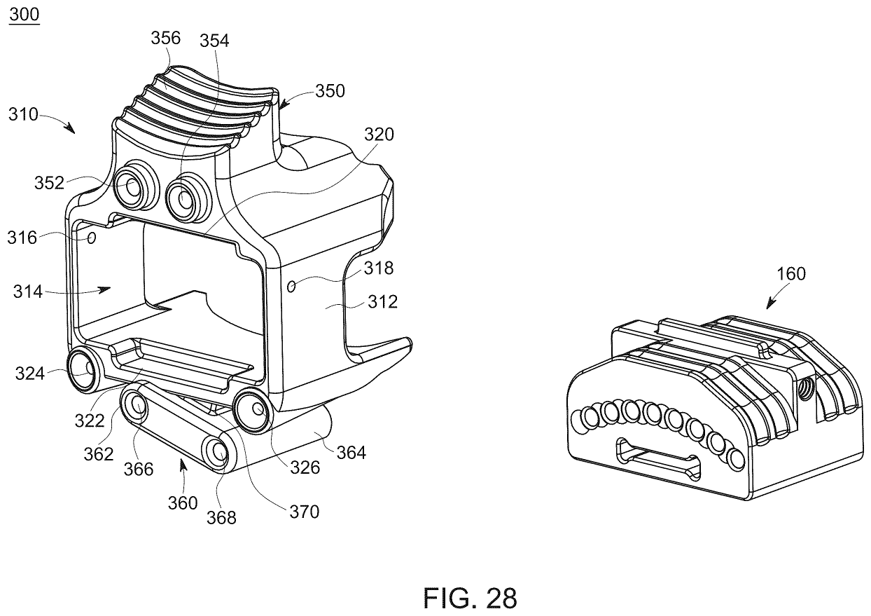

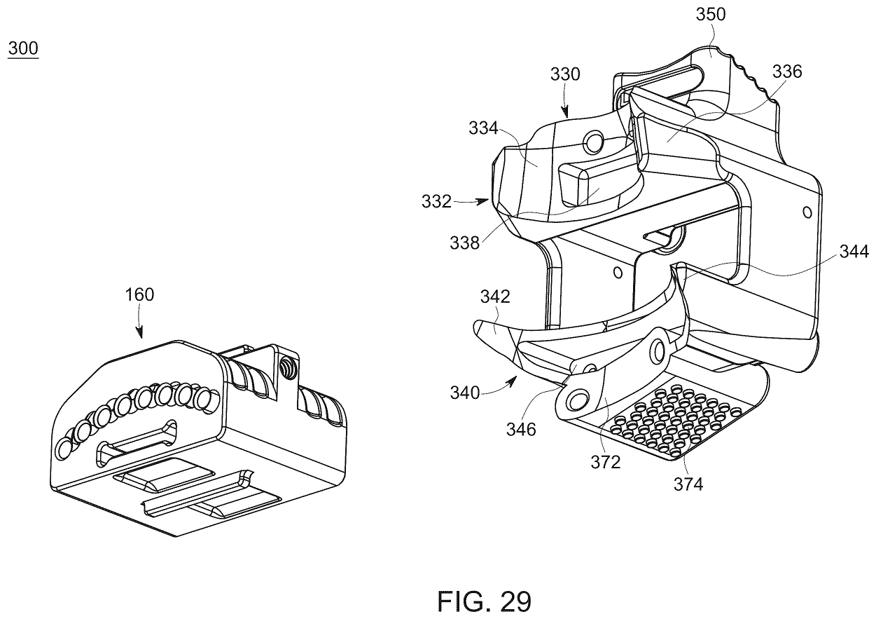

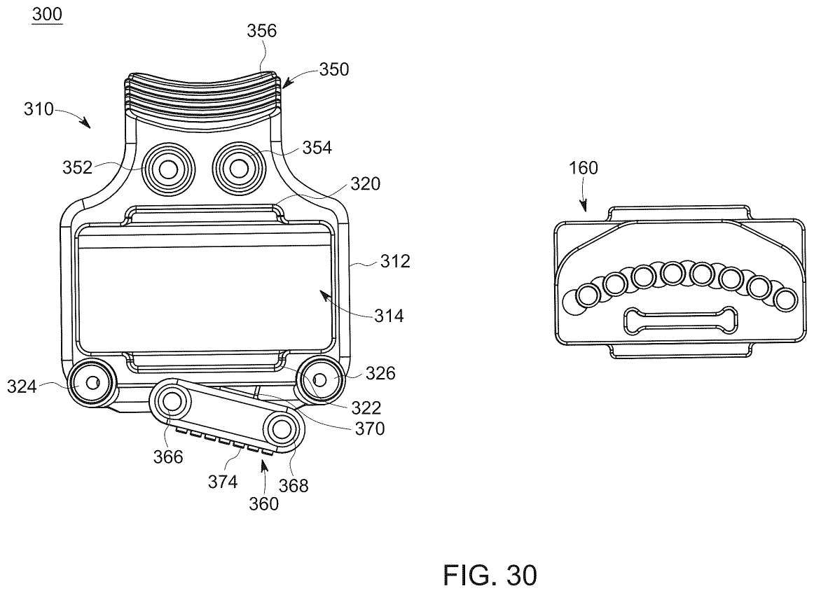

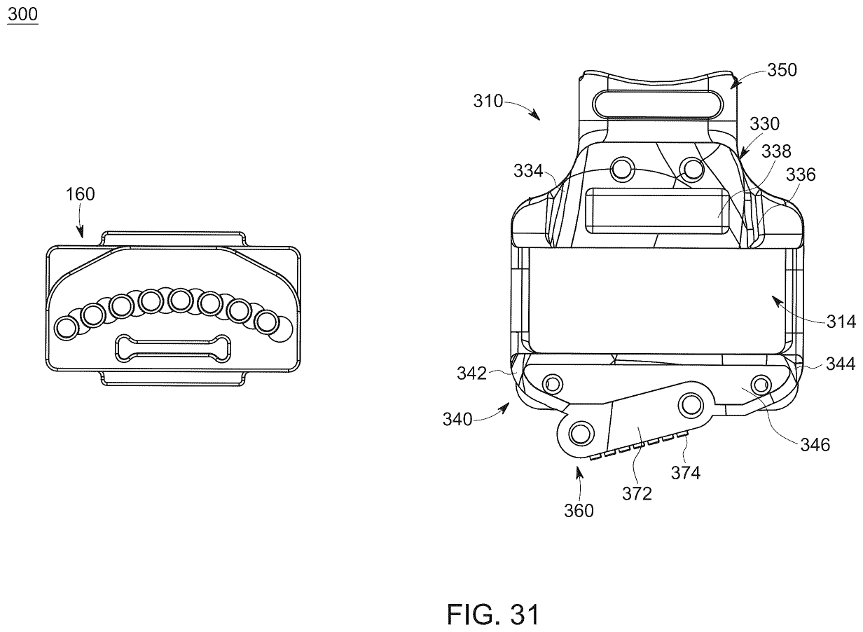

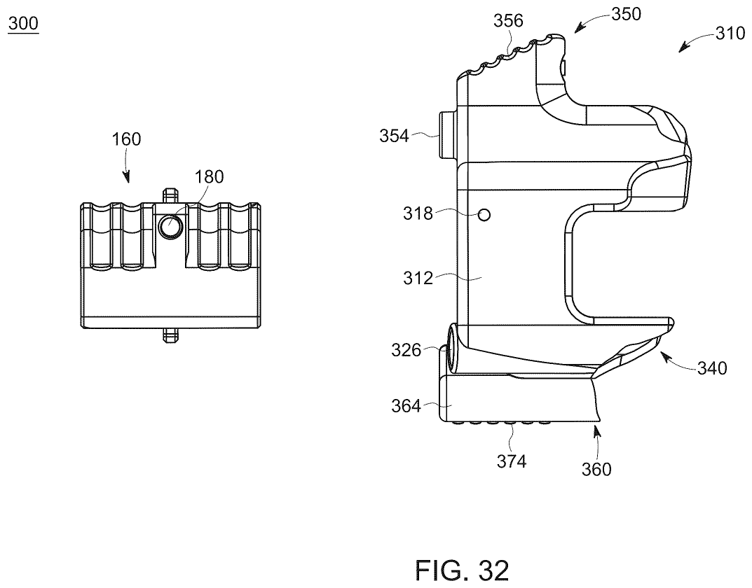

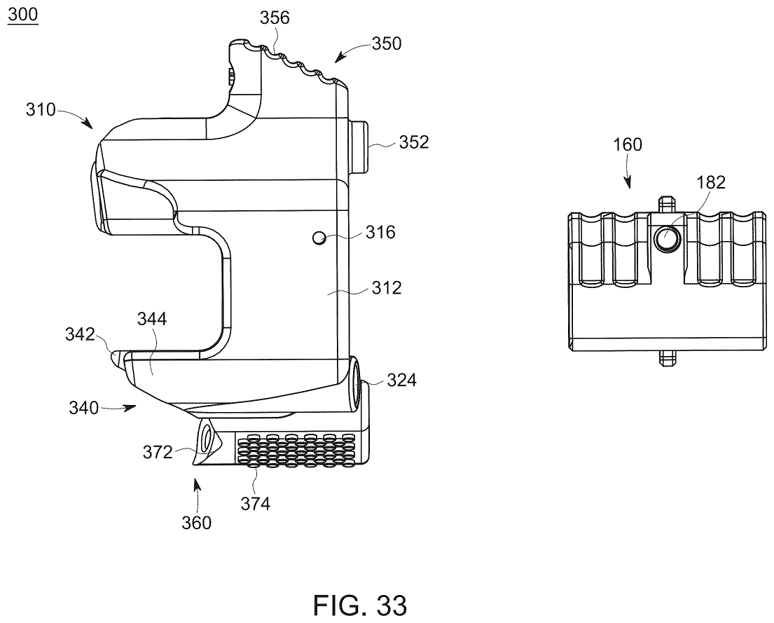

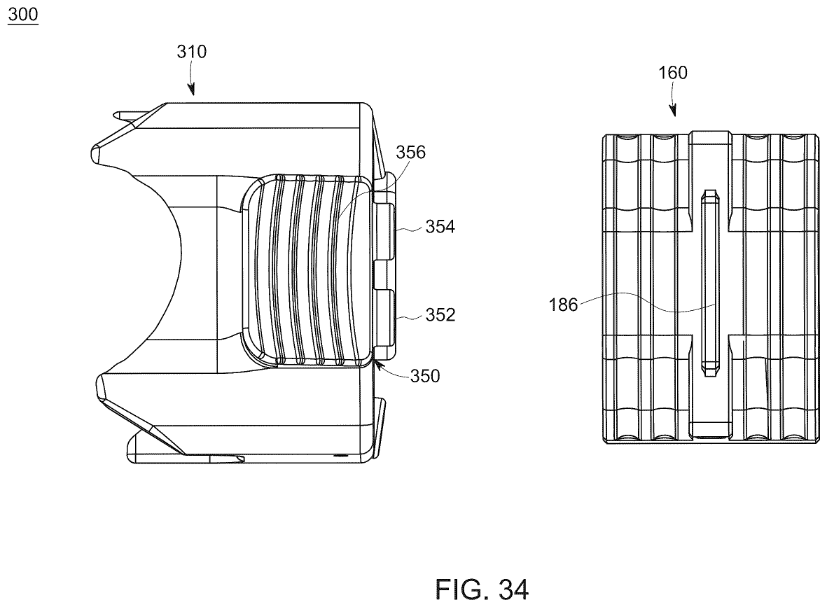

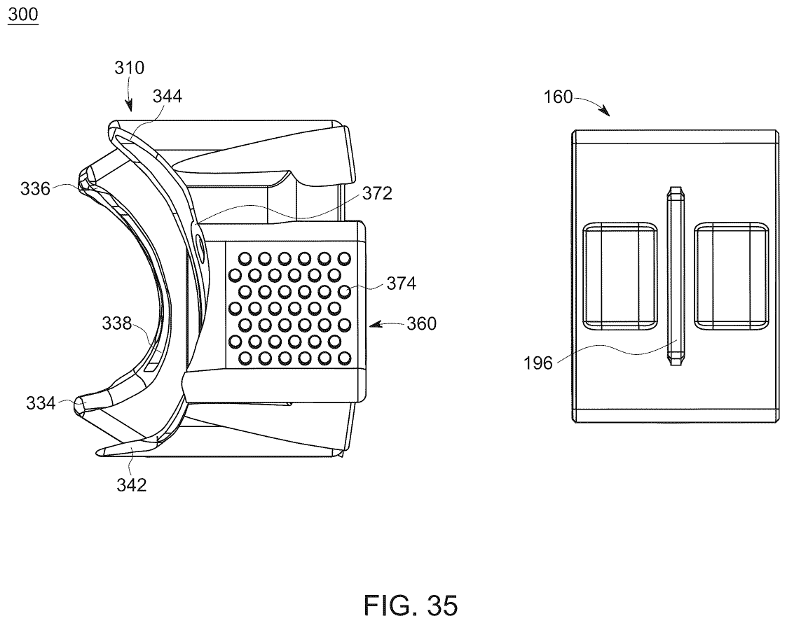

Referring now to FIGS. 18-35, a patient specific tibia osteotomy guide or guide system 300. The patient specific osteotomy guide 300 includes an alignment block 310 and the cut guide 160. The cut guide 160 is as described in greater detail above with reference to osteotomy guide 100 and will not be described again here for brevity's sake. The alignment block 310 includes a housing 312, a first alignment member 350 extending away from a top surface of the housing 312, and a second alignment member 360 extending away from a bottom surface of the housing 312.

As shown in FIGS. 18-35, the housing 312 includes a through hole 314 extending through the housing 312 from a first side or an anterior surface to a second side or a posterior surface. The through hole 314 may have, for example, a rectangular shape to correspond to the exterior shape of the cut block 160. The housing 112 may include at least one opening 316, 318 extending through the housing 312 from the through hole 314 to an exterior surface of the housing 312 on the medial and lateral sides of the housing 312. The at least one opening 316, 318 may include a first opening 316 extending through a first end of the housing 312 and a second opening 318 extending through a second end of the housing 312. The first end of the alignment block 310 is positioned opposite the second end of the alignment block 310. In addition, the first opening 316 is positioned opposite the second opening 318, for example, directly opposite. The housing 312 also includes at least one recess 320, 322 recessed into a front or anterior surface of the housing 312. The at least one recess 320, 322 may include a first recess 320 positioned at a top of the housing 312 and extending into a top surface of the through hole 314 and a second recess 322 positioned at a bottom of the housing 312 and extending into a bottom surface of the through hole 314. The housing 312 also includes at least one pin hole or alignment opening 324, 326 positioned near an inferior side of the housing 312 and extending through the housing 312 from a first or anterior side to a second or posterior side. The at least one pin hole 324, 326 may be, for example, a first pin hole 324 positioned on a first side of the second recess 322 and a second pin hole 326 positioned on a second side of the second recess 322. Each pin hole 324, 326 may be, for example, configured or sized and shaped to receive a temporary fastener, such as, a k-wire, olive wire, or the like.

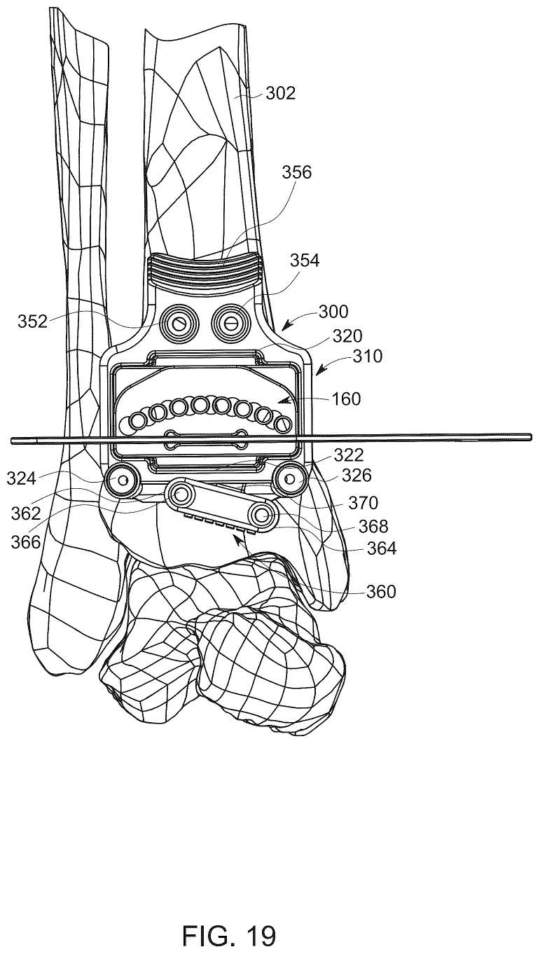

The alignment block 310 may also include a bone contacting surface 330 positioned on a back or posterior surface of the alignment block 310, as shown in FIGS. 21, 23, 29, and 31. The bone contacting surface 330 may be, for example, shaped to correspond to the shape of the bones on the anterior portion of the ankle bones. The bone contacting surface 330 may be customized to be patient specific based on imaging data obtained before creation of the alignment block 310. The bone contact surface 330 may include a first bone contacting portion 332 positioned near the superior side of the housing 312 and a second bone contacting portion 340 positioned near the inferior side of the housing 312. The through hole 314 may extend between the first bone contacting portion 332 and the second bone contacting portion 340. The first bone contacting portion 332 may include a first arm 334 extending away from the posterior side of the housing 312 on a first side and a second arm 336 extending away from the posterior side of the housing 312 on a second side. The arms 334, 336 may be, for example, formed or sized and shaped to wrap around a portion of the tibia 302, as shown in FIGS. 18 and 19. The first bone contact portion 332 may also include a first recessed region 338 positioned between the first and second arms 334, 336 and extending into the housing 312. The second bone contacting portion 340 may include a first arm 342 extending away from the posterior side of the housing 312 on a first side and a second arm 344 extending away from the posterior side of the housing 312 on a second side. The arms 342, 344 may be, for example, formed or sized and shaped to wrap around a portion of the tibia 302, as shown in FIGS. 18 and 19. The second bone contact portion 340 may also include a second recessed region 346 positioned between the first and second arms 342, 344 and extending into the housing 312. The second recessed region 346 may have, for example, a length between the first end and the second end of the housing 312 that is longer than the length of the first recessed region 338.

With continued reference to FIGS. 18-35, the first alignment member 350 is shown and includes at least one alignment opening 352, 354. The first alignment opening 352 may extend through the first alignment member 350 from a first or anterior side to a second or posterior side. The second alignment opening 354 may also extend through the first alignment member 350 from a first or anterior side to a second or posterior side. The alignment openings 352, 354 may extend through the posterior side of the osteotomy guide 300 above the first recessed region 338. The first alignment member 350 may also include a top surface 356 extending from the anterior surface to the posterior surface of the osteotomy guide 300 at, for example, an angle. The first alignment member 350 having a height on the anterior side smaller than the height on the posterior side. The top surface 356 may also be textured, for example, including at least one groove 358 extending into the top surface 356.

The second alignment member 360 is also shown in FIGS. 18-35. The second alignment member 360 may include a first end 362 and a second end 364. The second alignment member 360 may be coupled to the housing 312 with a connecting member 370. The second alignment member 360 may be coupled, for example, at an angle such that the first end 362 is positioned closer to the bottom surface of the housing 312 than the second end 364. Thus, the second alignment member 360 may extend from the housing 312 at an angle relative to the bottom surface of the housing 312 from the first end 362 to the second end 364. The second alignment member 360 may also include a first alignment opening 366 at the first end 362 and a second alignment opening 368 at the second end 364. The openings 366, 368 extend through the second alignment member 360 from an anterior or first side to a posterior or second side. Each opening 366, 368 may be, for example, configured or sized and shaped to receive a temporary fastener, such as, a k-wire, olive wire, or the like. The second alignment member 360 may also include a bone contacting surface 372 that is, for example, customized to match or correspond to the specific anatomy of the patient. Further, the second alignment member 360 may include a bottom surface 374 including, for example, a textured surface, such as a plurality of protrusions.

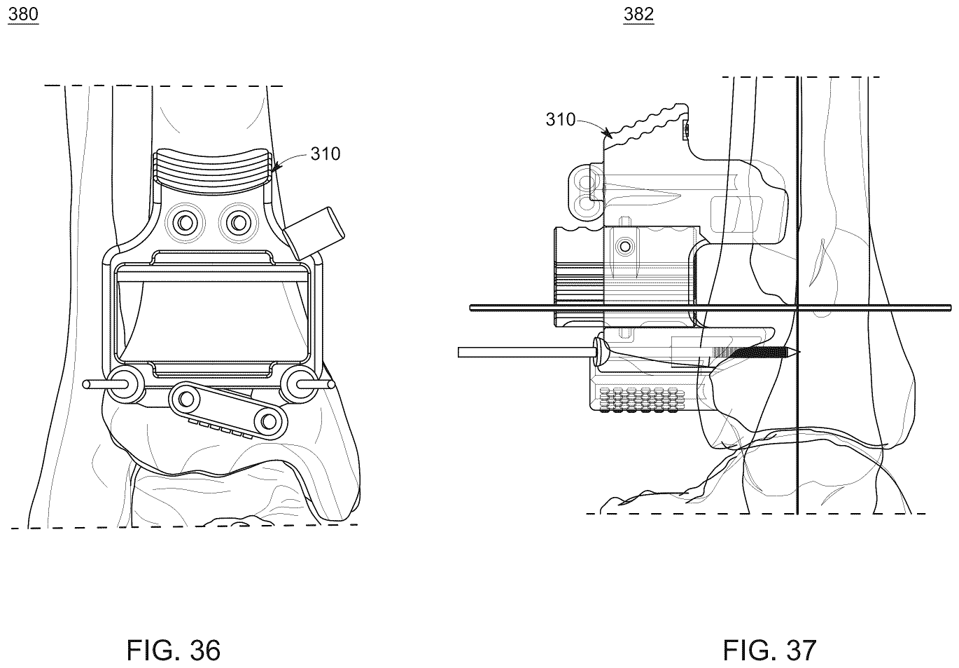

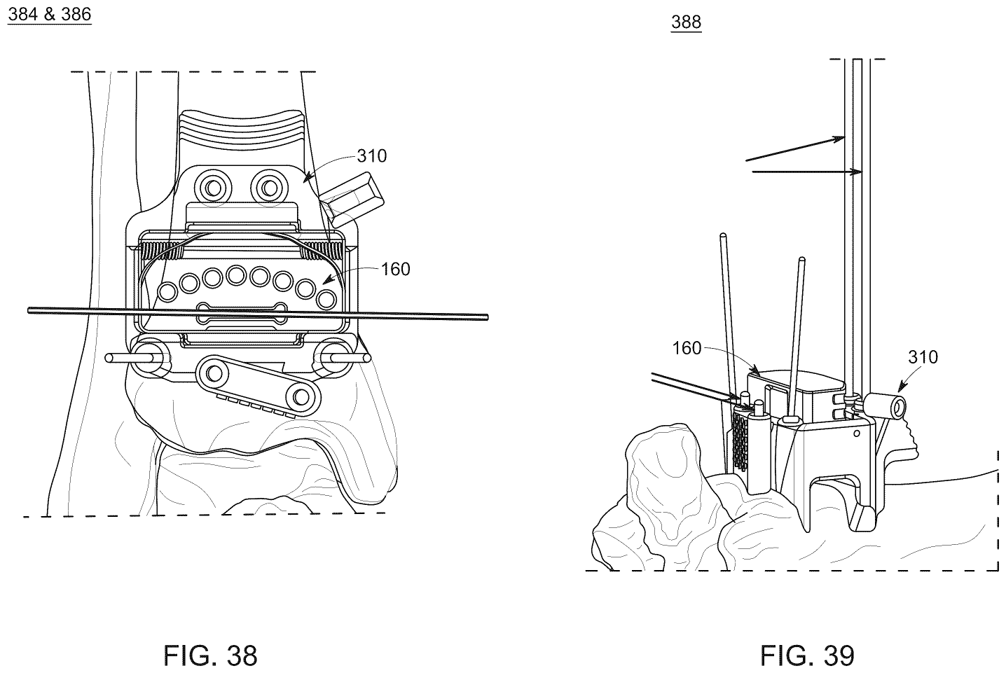

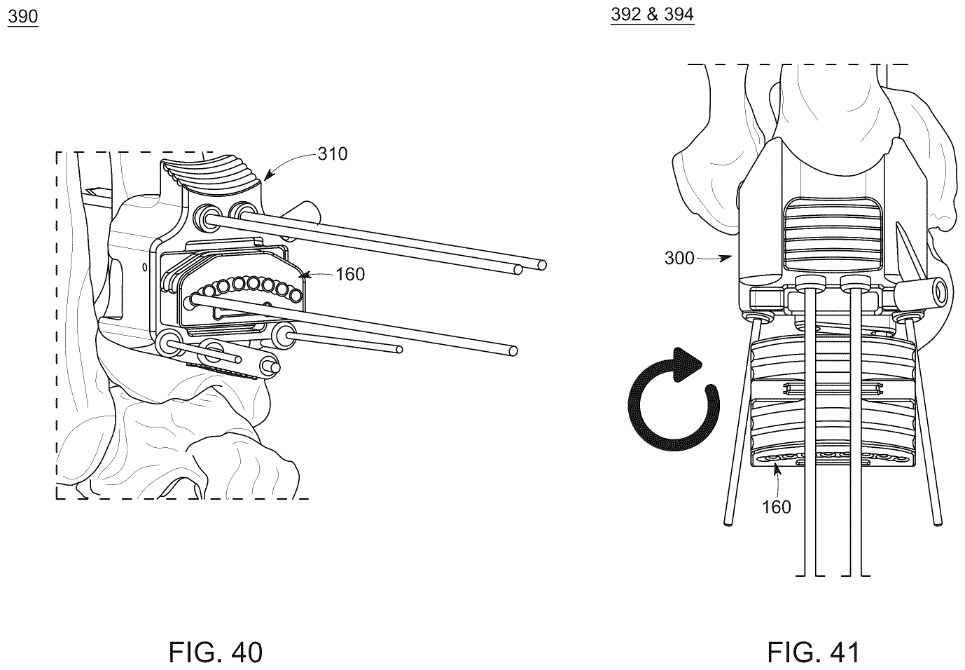

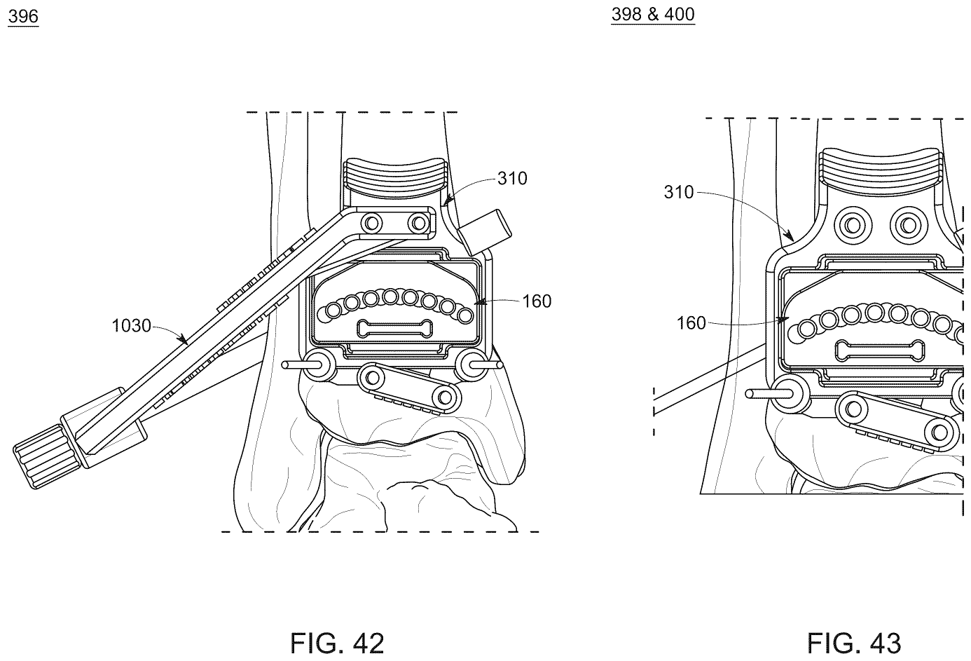

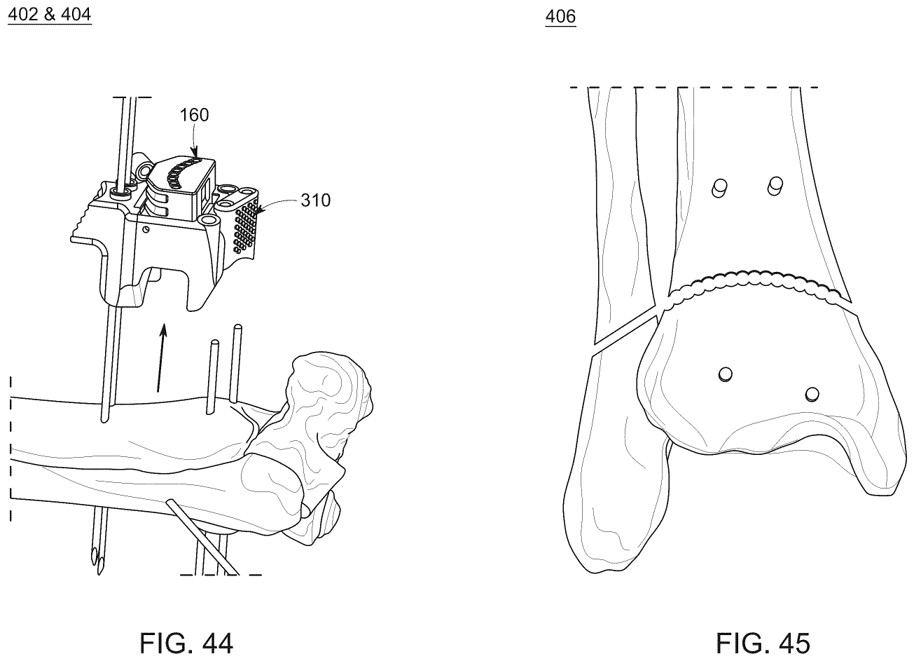

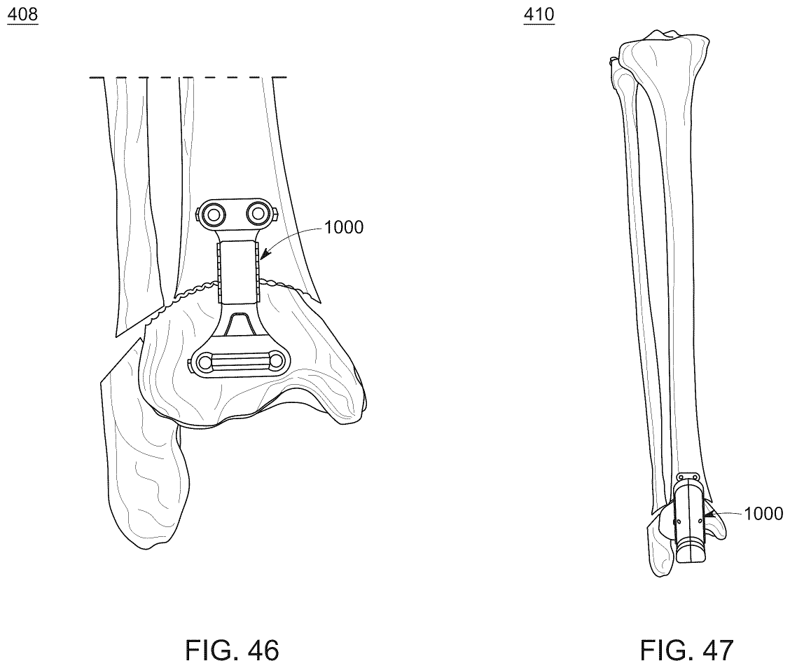

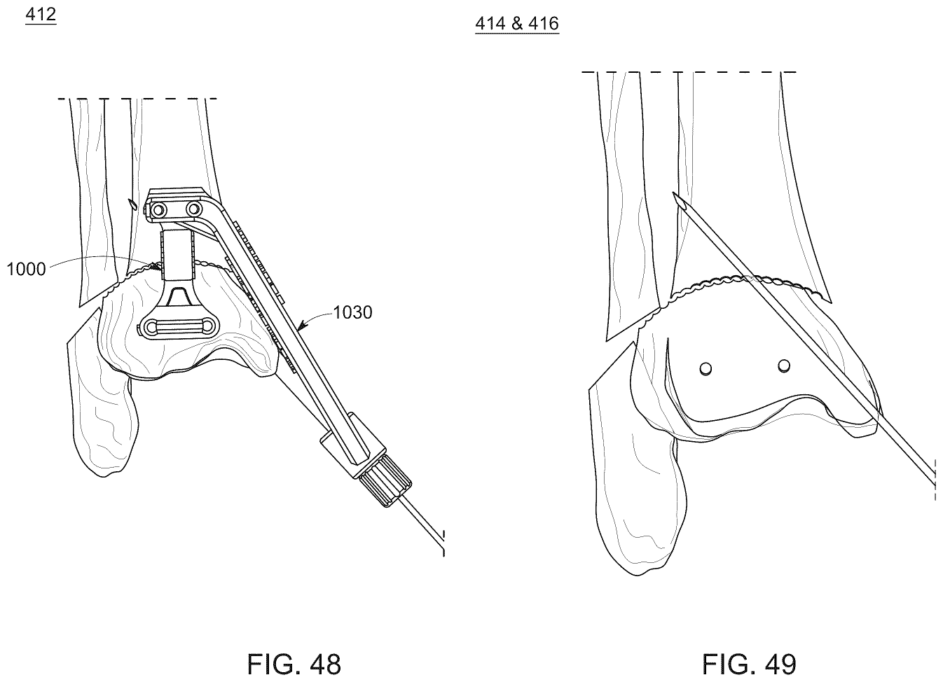

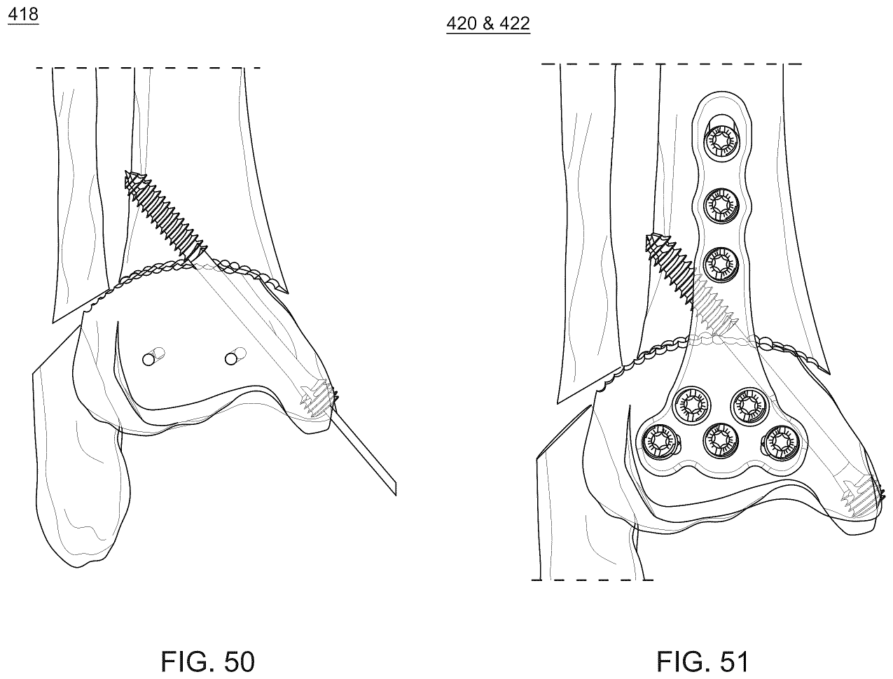

Referring now to FIGS. 36-51, a method for using the osteotomy guide 300 is shown. In some aspects, one or more of the components described herein and incorporated in the method of FIGS. 36-51 may be patient specific. For example, one or more of the components (e.g., the osteotomy guide 100, the alignment block 110, the cut block 160, etc.) may be generated (e.g., manufactured) with one or more features specific to an anatomy of the patient based on patient data. Further to the previous example, the alignment guide 100 may be generated to include a surface geometry matched (e.g., complimentary) to a surface of a bone of a patient to facilitate placement of the osteotomy guide 100 relative to the bone of the patient and thus guide a physician in performing various steps of the method of FIGS. 36-51 with respect to the anatomy of a specific patient. The method includes obtaining an alignment block and placing the alignment block onto the patient's bones 380. Next, temporary fixation is inserted through the alignment block and into the patient's bones 382. The temporary fixation may be, for example, olive wires, k-wires, or the like. The method may also include inserting a cut block into the alignment block 384 and verifying the position of the cut block 386. The position may be verified using, for example, an angel wing inserted into a slot in the cut block. The method may further include placing transfer pins into the proximal and distal alignment openings of the alignment block 388. The distal pins may be, for example, trimmed to the cannula height. Next, holes may be drilled through the cut block and into the bone 390. After the holes are drilled, the cut block may be removed and flipped over to the reverse side 392 for drilling the overlapping holes through the reverse side of the cut block and into the bone 394. A fibular guide may then be slid over the proximal wires 396. A slide pin tube may be inserted into the fibular guide before or after sliding the fibular guide over the proximal wires. Next, a fibular wire may be placed through the fibula using the fibular guide 398. Once the fibular wire is placed, the pin tube and fibular guide may be removed from the alignment block 400. Then, the temporary fixation, for example, olive wires, may be removed 402 and the alignment block and cut guide may be slid off the k-wires 404. After removal of the osteotomy guide, the osteotomy may be completed and the fibular wire followed to make the fibula osteotomy 406. The osteotomy may be completed by, for example, connecting or cleaning up the drilled or cut openings. A correction guide may then be used to correct the alignment of the bones 408. Once the correction guide is in place, the position or alignment of the bones may be verified 410. The verification of the bone position may be done by, for example, a laser. Next, a tibia screw guide may be slid over the proximal wires in the tibia 412. The tibia screw guide may be inserted with or without the slide pin tube inserted into the guide. Once the tibia screw guide and slide pin tube are in place, a wire may be inserted through the tibia to hold the correction 414. After the wire is placed through the tibial bone portions, the tibia screw guide, correction guide, and transfer pins may be removed 416. A crossing screw may optionally be inserted over the wire placed through the tibial bone portions to secure the tibial bone pieces 418. Alternatively or in addition to the crossing screw, a permanent fixation device may be placed over the bone portions of the tibia 420 and secured in place 422. The permanent fixation device may be, for example, a bone plate secured with bone screws.

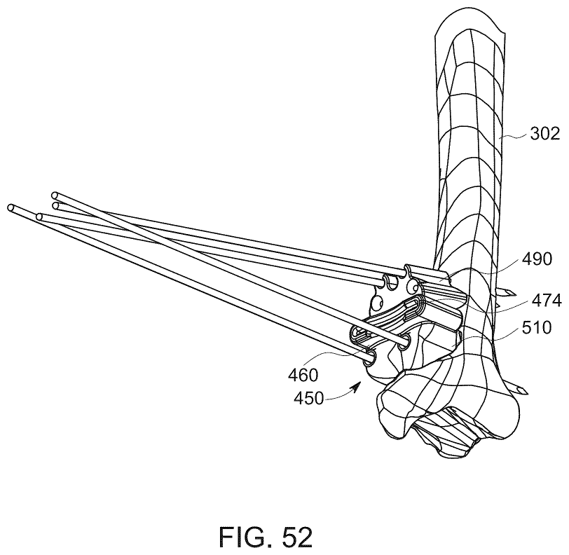

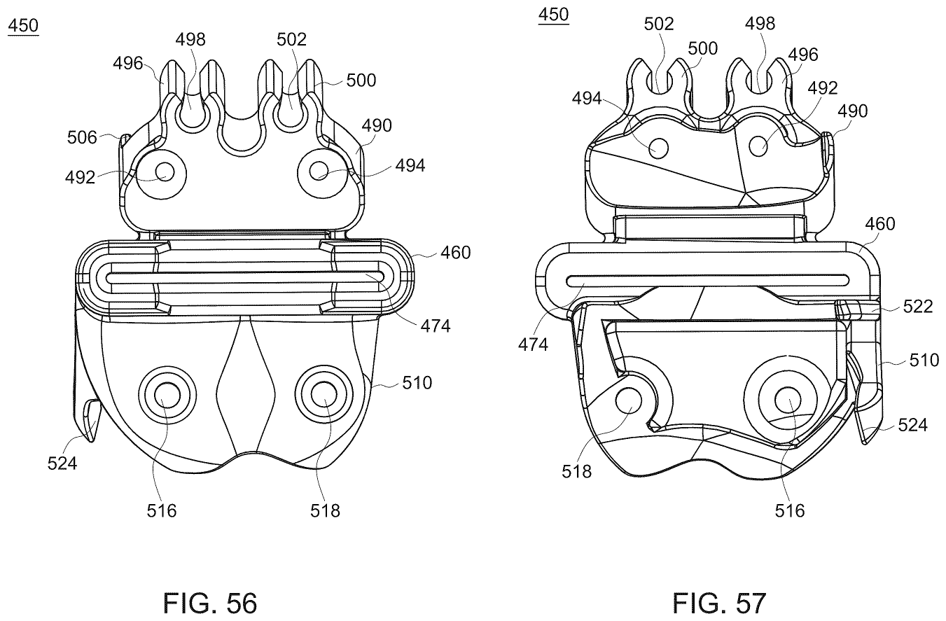

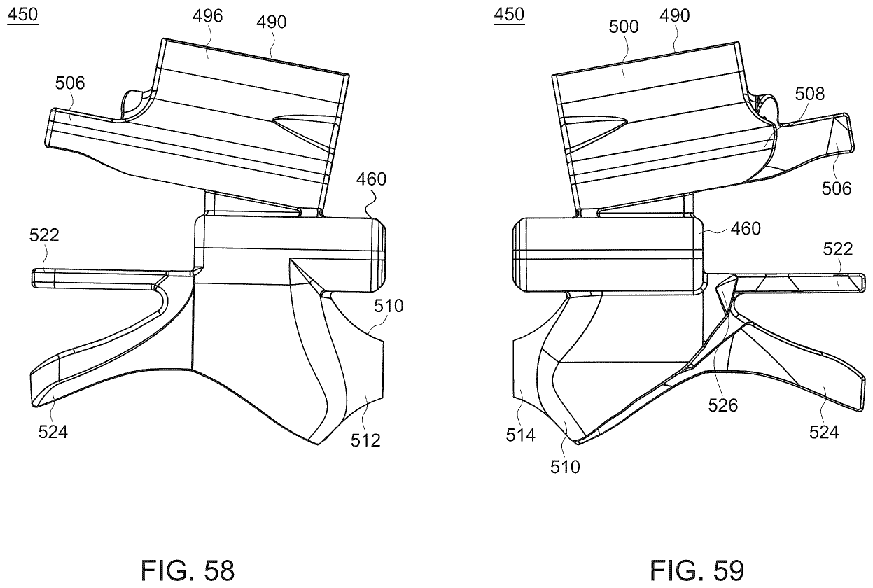

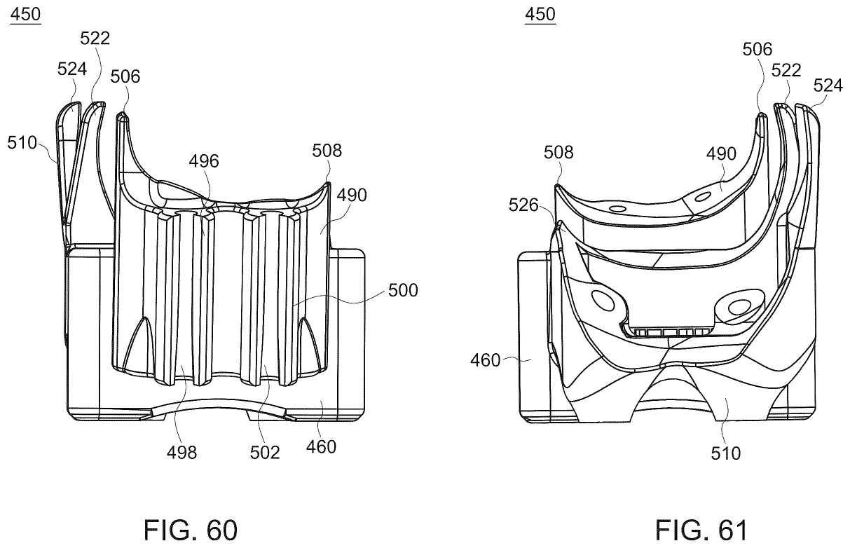

Referring now to FIGS. 52-61, another osteotomy guide 450 is shown. The osteotomy guide 450 is configured or sized and shaped for use with, for example, a medial approach. The osteotomy guide 450 is also configured or sized and shaped for an opening osteotomy. The osteotomy guide 450 includes a body 460, first alignment member 490 extending away from a superior side 470 of the body 460, and a second alignment member 510 extending away from an inferior side 472 of the body 460. Although shown as a single piece, it is also contemplated that the guide 450 may be a two piece construct coupled together at a connecting member 480. The body 460 includes a first side 462 opposite a second side 464, a first end 466 opposite a second end 468, and a superior side 470 opposite an inferior side 472. The body 460 also includes a slot 474 extending through the body 460 from the first side 462 to the second side 464. The slot 474 may also extend between a position near the first end 466 and a position near the second end 468. The body 460 may further include a first recess 476 extending into the body 460 from the first side 462 on a superior side 470 of the body 460. The body 460 also includes a second recess 478 extending into the body 460 from the first side 462 on an inferior side 472 of the body 460. The first and second recesses 476, 478 may extend into the body 460 in, for example, a curved or arced shape toward to second side 464 of the body 460. The osteotomy guide 450 may also include a connecting member 480 coupled to and extending from the superior side 470 of the body 460. The connecting member 480 may be, for example, a wedge shape with the smaller portion of the wedge position toward the first side 462 of the body 460. The larger portion of the wedge may be positioned at the second side 464 of the body 460 to angle the first alignment member 490 relative to the superior side 470 of the body 460.

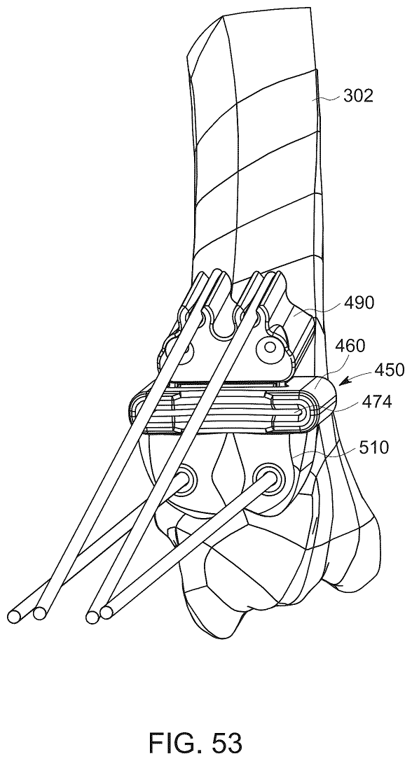

As shown in FIGS. 52-61, the first alignment member 490 may include a first pin hole or alignment opening 492 and a second pin hole or alignment opening 494 extending through the first alignment member 490 from a first side to a second or bone contacting side. The first and second pin holes 492, 494 may, for example, converge or be angled towards each other as the pin holes 492, 494 extend through the first alignment member 490. The first alignment member 490 also includes a first extension 496 and a second extension 500 extending away from a top of the first alignment member 490 above the pin holes 492, 494. The first and second extensions 496, 500 extend from the first side to the second side of the first alignment member 490. The first extension 496 also includes a first channel 498 extending into the first extension 496 from a top surface of the first alignment member 490 towards a bottom surface of the osteotomy guide 450. The second extension 500 also includes a second channel 502 extending into the second extension 500 from a top surface of the first alignment member 490 towards the bottom surface of the osteotomy guide 450. The channels 498, 502 may be, for example, configured or sized and shaped to receive and retain a temporary fixator, such as, a k-wire. As the first alignment member 490 is angled relative to a top surface of the body 460, the extensions 496, 500 are also angled as the extensions 496, 500 extend from the first side to the second side of the first alignment member 490. Referring now to FIGS. 55, 57, 60 and 61, a first bone contacting portion 504 of the first alignment member 490 is shown. The first bone contacting portion 504 includes a first arm 506 extending away from a first side of the first alignment member 490 and a second arm 508 extending away from a second side of the first alignment member 490. The first and second arms 506, 508 are, for example, configured or sized and shaped to engage a portion of the tibia, as shown in FIGS. 52 and 53.

Referring now to FIGS. 51-61, the second alignment member 510 is shown. The second alignment member 510 includes a first protrusion 512 and a second protrusion 514 each extending away from a first side of the second alignment member 510. The first protrusion 512 is positioned between the first end and a midpoint of the second alignment member 510, while the second protrusion 514 is positioned between the midpoint and a second end of the second alignment member 510. A first alignment opening 516 extends through the first protrusion 512 to the second side of the second alignment member 510. A second alignment opening 518 extends through the second protrusion 514 to the second side of the second alignment member 510. The alignment openings 516, 518 may be, for example, configured or sized and shaped to receive a temporary fixator, such as, a k-wire or the like.

As shown in FIGS. 55, 57, 60 and 61, the second alignment member 510 also includes a second bone contacting portion 520 positioned on the second side of the second alignment member 510. The second bone contacting portion 520 includes a first arm 522 and a second arm 524 extending away from the first side of the second alignment member 510. The first arm 522 is positioned superior to and spaced apart from the second arm 524. The first arm 522 may have a width and length smaller than the width and length of the second arm 524. The second bone contacting portion 520 may also include a third arm 526 extending away from the second side of the second alignment member 510. The third arm 526 may have, for example, a length shorter than both the first and second arms 522, 524. The second bone contacting portion 520 may also include a recessed region 528 extending into the second alignment member 510 from the second side. The recessed region 528 may include a first projection 530 surrounding the first alignment opening 516 and a second projection 532 surrounding the second alignment opening 518. The projections 530, 532 may extend away from the surface of the recessed region 528.

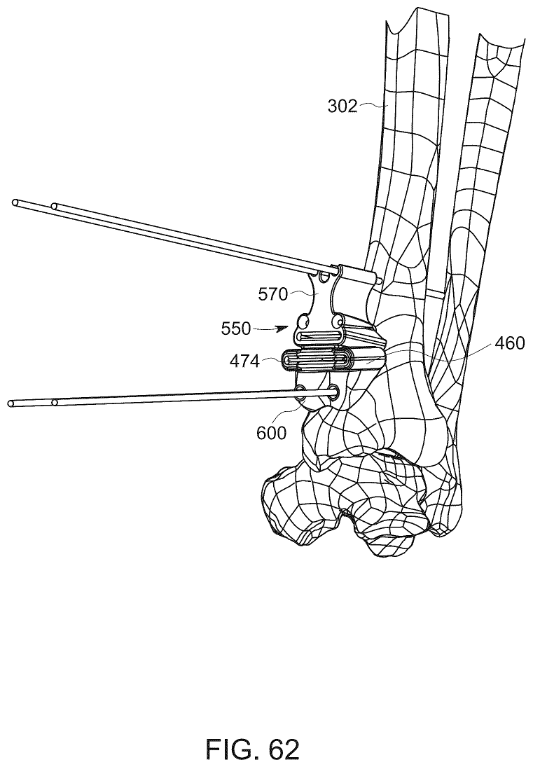

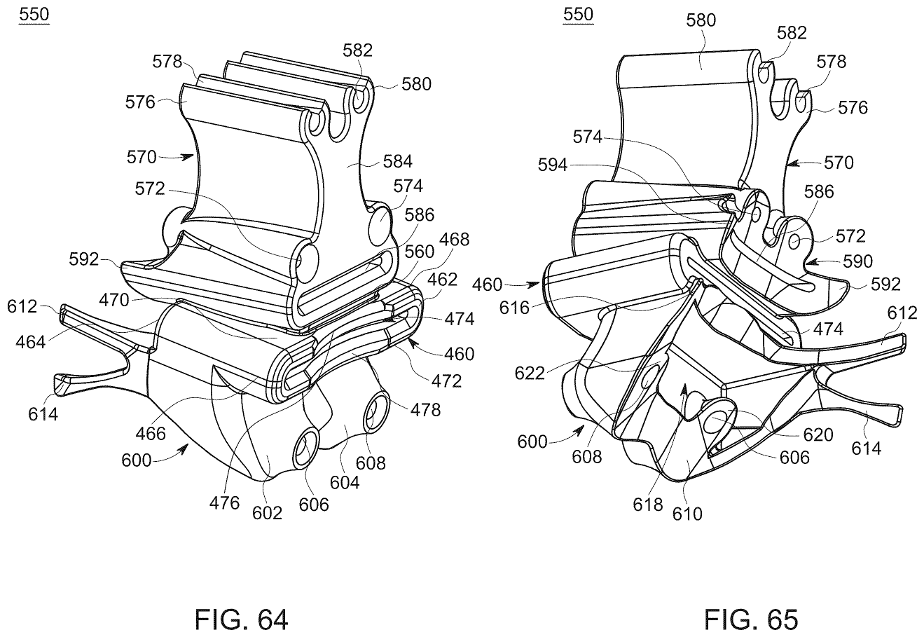

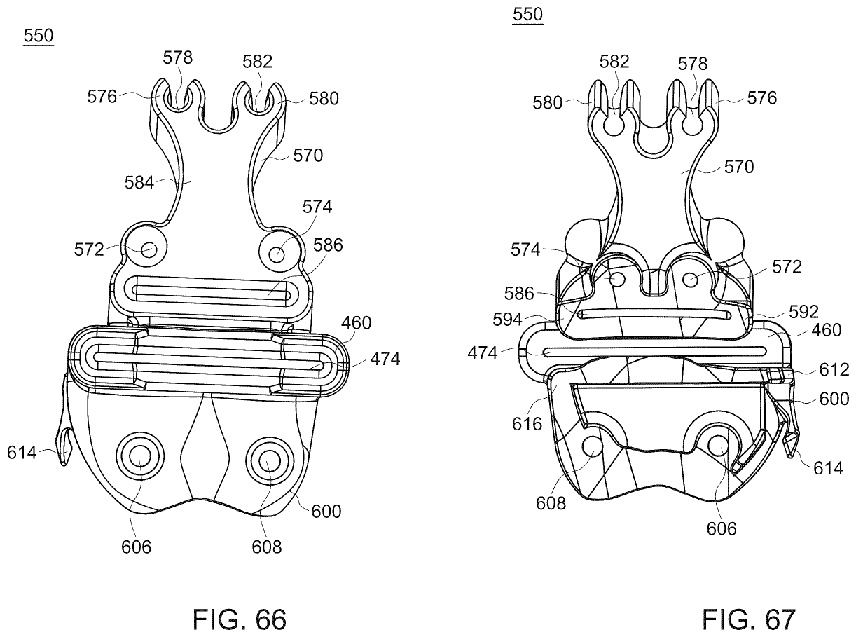

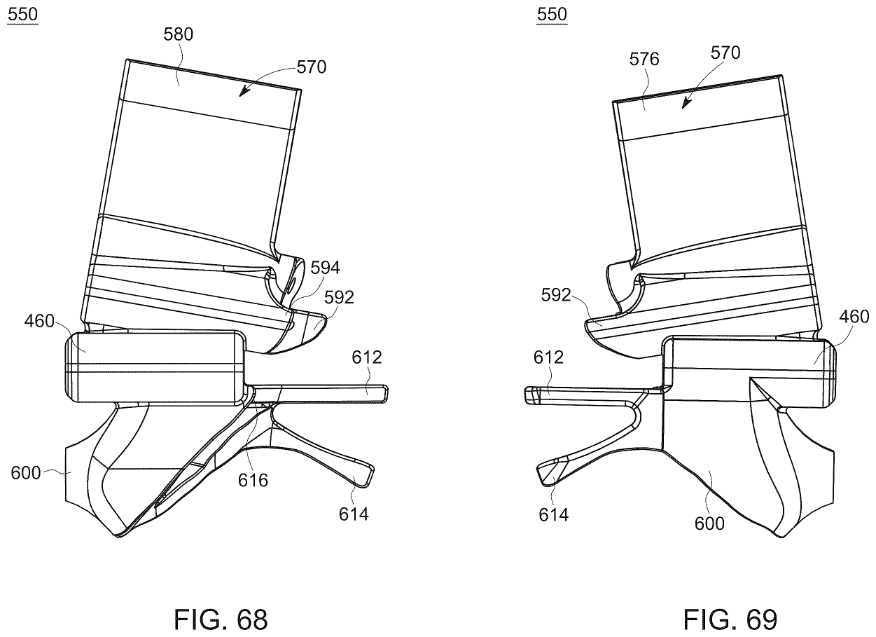

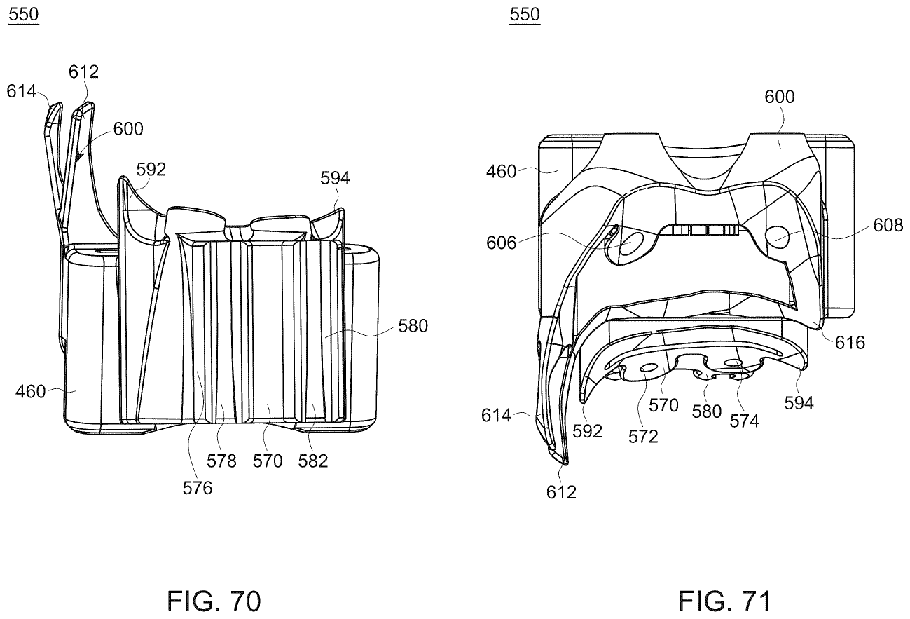

Referring now to FIGS. 62-71, another osteotomy guide 550 is shown. The osteotomy guide 550 is configured or sized and shaped for use with, for example, a medial approach. The osteotomy guide 550 is also configured or sized and shaped for a closing osteotomy. The osteotomy guide 550 includes a body 460, first alignment member 570 extending away from a superior side 470 of the body 460, and a second alignment member 600 extending away from an inferior side 472 of the body 460. Although shown as a single piece, it is also contemplated that the guide 550 may be a two piece construct coupled together at a connecting member 560. The body 460 is as described above with reference to osteotomy guide 450 and which will not be described again here for brevity's sake. The osteotomy guide 550 may also include a connecting member 560 coupled to and extending from the superior side 470 of the body 460. The connecting member 560 may be, for example, a wedge shape with the larger portion of the wedge position at the first side 462 of the body 460. The smaller portion of the wedge may be positioned toward the second side 464 of the body 460 to angle the first alignment member 570 relative to the superior side 470 of the body 460.

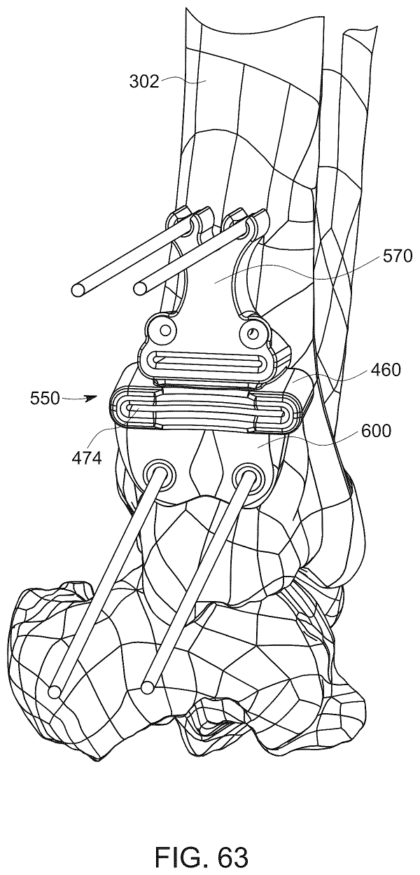

As shown in FIGS. 62-71, the first alignment member 570 may include a first pin hole or alignment opening 572 and a second pin hole or alignment opening 574, each extending through the first alignment member 570 from a first side to a second or bone contacting side. The first and second pin holes 572, 574 may, for example, converge or be angled towards each other as the pin holes 572, 574 extend through the first alignment member 570. The first alignment member 570 also includes a first extension 576 and a second extension 580 extending away from a top of the first alignment member 570 above the pin holes 572, 574. The first alignment member 570 may also include a neck portion 584 extending between the pin holes 572, 574 and the extensions 576, 580. The first and second extensions 576, 580 extend from the first side to the second side of the first alignment member 570. The first extension 576 also includes a first channel 578 extending into the first extension 576 from a top surface of the first alignment member 570 towards the neck portion 584 and/or a bottom surface of the osteotomy guide 550. The second extension 580 also includes a second channel 582 extending into the second extension 580 from a top surface of the first alignment member 570 towards the neck portion 584 and/or the bottom surface of the osteotomy guide 550. The channels 578, 582 may be, for example, configured or sized and shaped to receive and retain a temporary fixator, such as, a k-wire. As the first alignment member 570 is angled relative to a top surface of the body 460, the extensions 576, 580 are also angled as the extensions 576, 580 extend from the first side to the second side of the first alignment member 570. Referring now to FIGS. 65, 67, 70 and 71, a first bone contacting portion 590 of the first alignment member 570 is shown. The first bone contacting portion 590 includes a first arm 592 extending away from a first side of the first alignment member 570 and a second arm 594 extending away from a second side of the first alignment member 570. The first and second arms 592, 594 are, for example, configured or sized and shaped to engage a portion of the tibia, as shown in FIGS. 62 and 63.

Referring now to FIGS. 62-71, the second alignment member 600 is shown. The second alignment member 600 includes a first protrusion 602 and a second protrusion 604, each extending away from a first side of the second alignment member 600. The first protrusion 602 is positioned between the first end and a midpoint of the second alignment member 600, while the second protrusion 604 is positioned between the midpoint and a second end of the second alignment member 600. A first alignment opening 606 extends through the first protrusion 602 to the second side of the second alignment member 600. A second alignment opening 608 extends through the second protrusion 604 to the second side of the second alignment member 600. The alignment openings 606, 608 may be, for example, configured or sized and shaped to receive a temporary fixator, such as, a k-wire or the like.

As shown in FIGS. 65, 67, 70 and 71, the second alignment member 600 also includes a second bone contacting portion 610 positioned on the second side of the second alignment member 600. The second bone contacting portion 610 includes a first arm 612 and a second arm 614 extending away from the first side of the second alignment member 600. The first arm 612 is positioned superior to and spaced apart from the second arm 614. The first arm 612 may have a width and length similar to the width and length of the second arm 614. The second bone contacting portion 610 may also include a third arm 616 extending away from the second side of the second alignment member 600. The third arm 616 may have, for example, a length shorter than both the first and second arms 612, 614. The second bone contacting portion 610 may also include a recessed region 618 extending into the second alignment member 600 from the second side. The recessed region 618 may include a first projection 620 surrounding the first alignment opening 606 and a second projection 622 surrounding the second alignment opening 608. The projections 620, 622 may extend away from the surface of the recessed region 618.

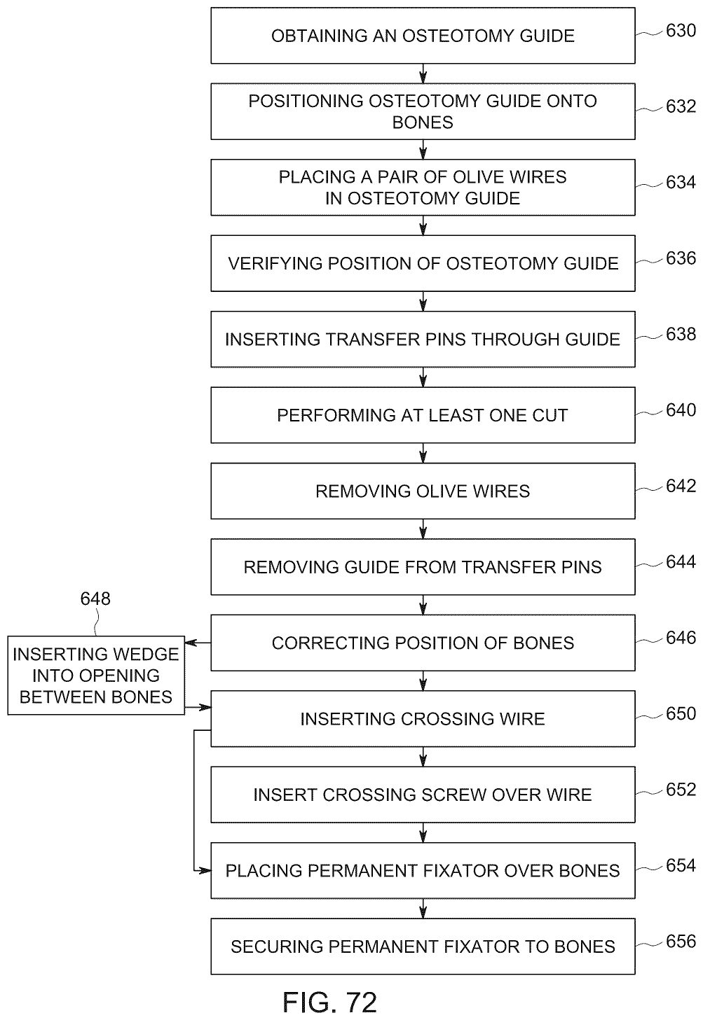

Referring now to FIG. 72, a method for using the osteotomy guides 450, 550 is shown. In some aspects, one or more of the components described herein and incorporated in the method of FIG. 72 may be patient specific. For example, one or more of the components (e.g., the osteotomy guides 450, 550, etc.) may be generated (e.g., manufactured) with one or more features specific to an anatomy of the patient based on patient data. Further to the previous example, the alignment guide 450, 550 may be generated to include a surface geometry matched (e.g., complimentary) to a surface of a bone of a patient to facilitate placement of the osteotomy guide 450, 550 relative to the bone of the patient and thus guide a physician in performing various steps of the method of FIG. 72 with respect to the anatomy of a specific patient. The method includes obtaining an osteotomy guide based on the size of the desired wedge to be inserted or take out 630. After the osteotomy guide is selected, the guide may be positioned onto the bone 632, such as shown in FIGS. 52-53 for guide 450 and FIGS. 62-63 for guide 550. Next, temporary fixators, such as, olive wires may be inserted into the pin holes 634. Once the guide is temporarily fixed to the tibia, the position of the guide may be verified using, for example, an angel wing inserted into a slot in the body of the osteotomy guide 636. After the position is verified, transfer pins or k-wires may be inserted into the proximal channels and distal alignment openings 638. Next, at least one cut may be performed using the at least one slot of the osteotomy guides 640. For the guide 450, a single cut will be made using the slot in the body. While, for the guide 550, two cuts may be made using the slot in the body and the slot in the first alignment member to allow for removal of a wedge portion of bone. After the one or more cuts are complete, the olive wires may be removed 642 and the guide may be removed from the transfer pins 644. Then, the transfer pins may be used to assist with correcting the position of the bone portions 646. If performing an opening procedure, then a wedge may be prepared and inserted into the opening between the two bone portions 648. After the bone portions have been positioned in the desired orientation, then a crossing wire may be inserted across the two bone portions 650. A crossing screw may then be optionally inserted over the crossing wire and into the two bone portions 652. Then, a permanent fixator may be placed over the bone portions 654 and the permanent fixator secured to the bone portions 656. The permanent fixator may be, for example, a bone plate.

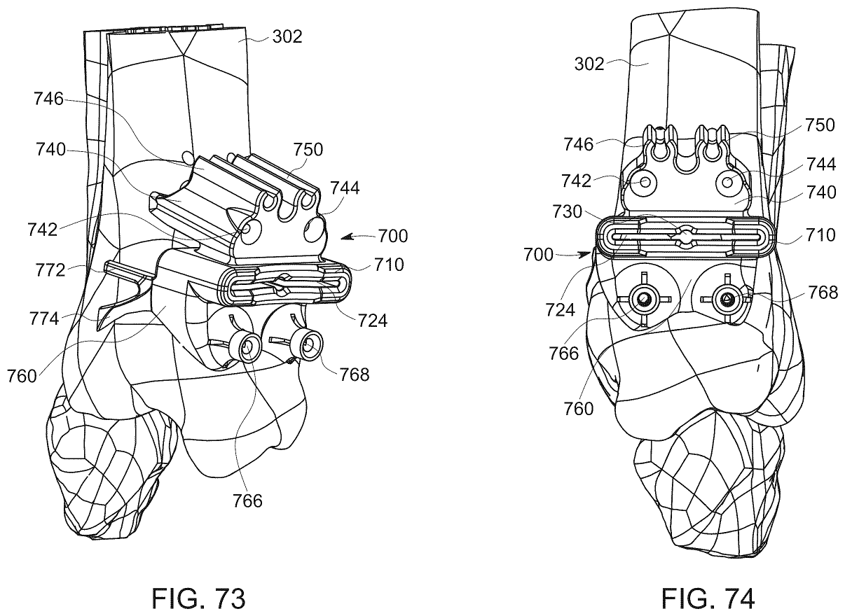

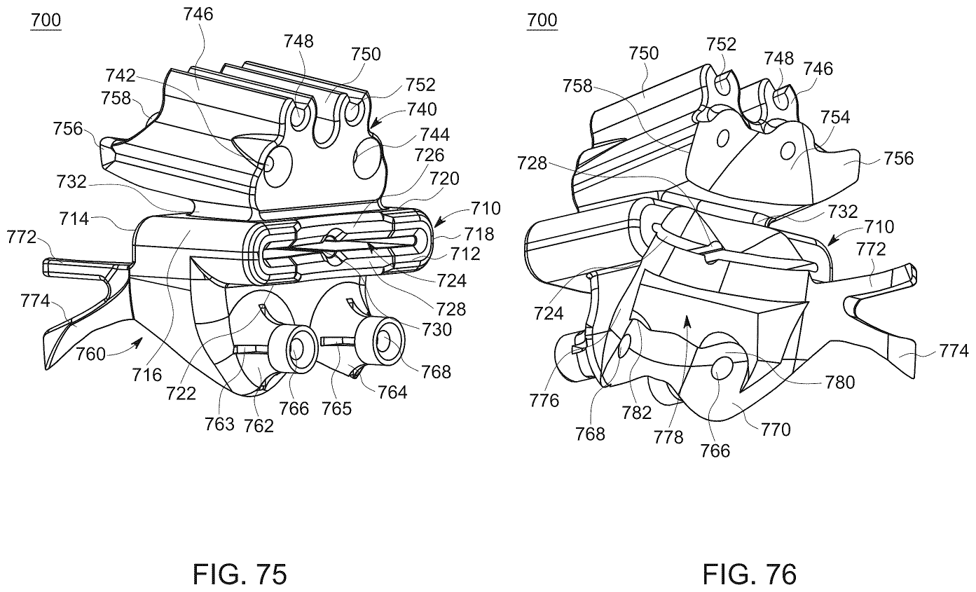

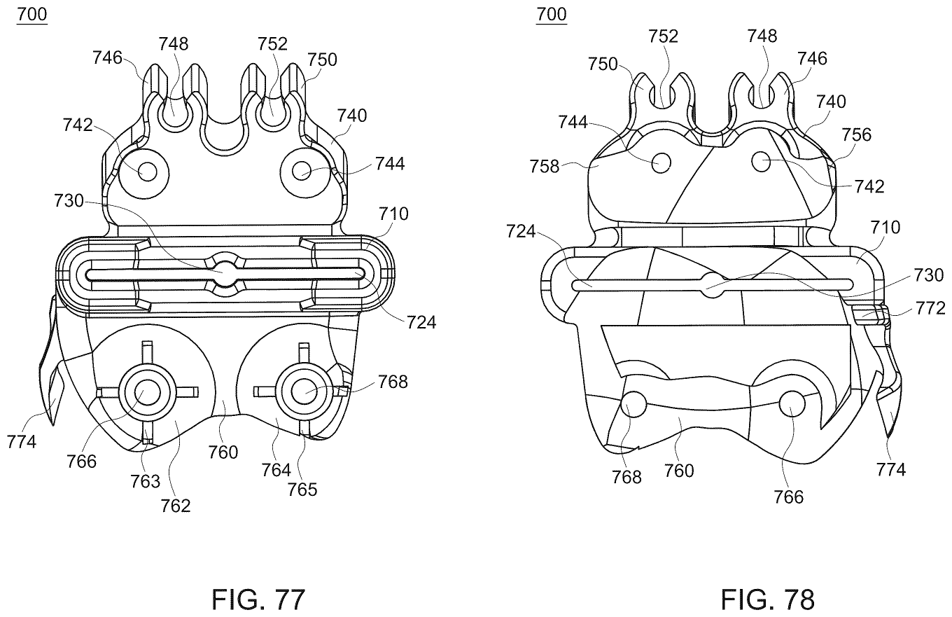

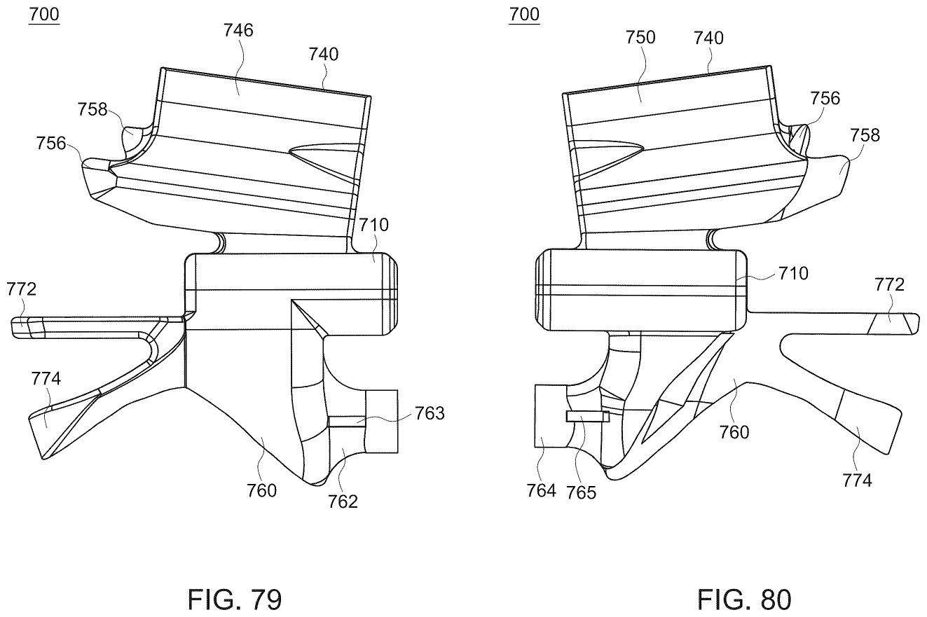

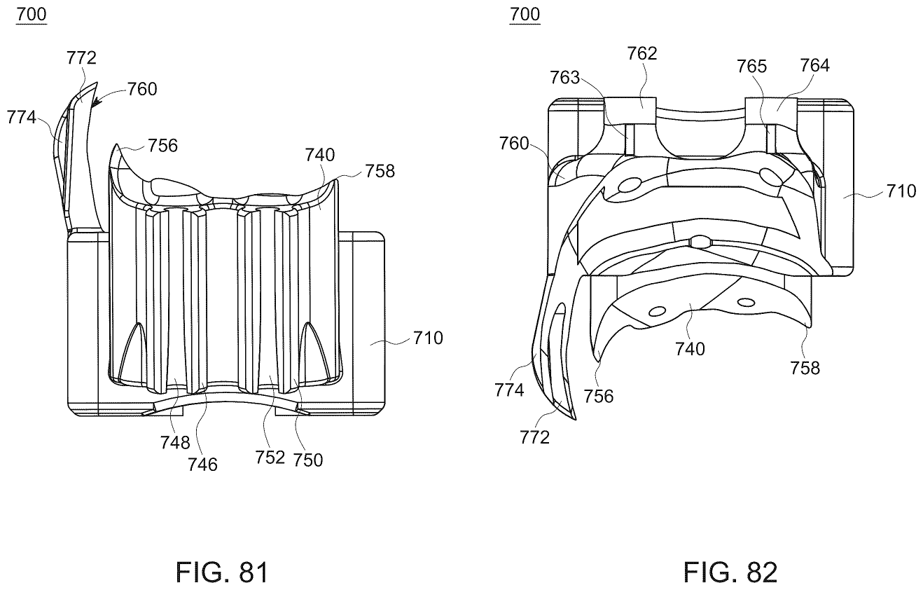

Referring now to FIGS. 73-82, another osteotomy guide 700 is shown. The osteotomy guide 700 is configured or sized and shaped for use with, for example, a medial approach. The osteotomy guide 700 is also configured or sized and shaped for an opening osteotomy. The osteotomy guide 700 includes a body 710, a first alignment member 740 extending away from a superior side 720 of the body 710, and a second alignment member 760 extending away from an inferior side 722 of the body 710. Although shown as a single piece, it is also contemplated that the guide 700 may be a two piece construct coupled together at a connecting member 732. The body 710 includes a first side 712 opposite a second side 714, a first end 716 opposite a second end 718, and a superior side 720 opposite an inferior side 722. The body 710 also includes a slot 724 extending through the body 710 from the first side 712 to the second side 714. The slot 724 may also extend between a position near the first end 716 and a position near the second end 718. The body 710 may further include a first recess 726 extending into the body 710 from the first side 712 on a superior side 720 of the body 710. The body 710 also includes a second recess 728 extending into the body 710 from the first side 712 on an inferior side 722 of the body 710. The first and second recesses 726, 728 may extend into the body 710 in, for example, a curved or arced shape toward to second side 714 of the body 710. The guide 700 may also include a cut slot relief 730 position at or near a midpoint of the slot 724. The relief 730 may have, for example, a round or circular shape and extend from a first side 712 of the body 710 to a second side 714 of the body 710. The diameter of the relief 730 may be, for example, larger than the height of the slot 724. The relief 730 may also be configured or sized and shaped to receive a drive pin or the like. A first portion of the relief 730 may extend into a portion of the top surface of the slot 724 and a second portion of the relief 730 may extend into a portion of the bottom surface of the slot 724.

The osteotomy guide 700 may also include a connecting member 732 coupled to and extending from the superior side 720 of the body 710. The connecting member 732 may be, for example, a wedge shape with the smaller portion of the wedge position at or toward the first side 712 of the body 710. The larger portion of the wedge may be positioned at or toward the second side 714 of the body 710 to angle the first alignment member 740 relative to the superior side 720 of the body 710.

As shown in FIGS. 73-82, the first alignment member 740 may include a first pin hole or alignment opening 742 and a second pin hole or alignment opening 744 extending through the first alignment member 740 from a first side to a second or bone contacting side. The first and second pin holes 742, 744 may, for example, converge or be angled towards each other as the pin holes 742, 744 extend through the first alignment member 740. The first alignment member 740 also includes a first extension 746 and a second extension 750 extending away from a top of the first alignment member 740 above the pin holes 742, 744. The first and second extensions 746, 750 extend from the first side to the second side of the first alignment member 740. The first extension 746 also includes a first channel 748 extending into the first extension 746 from a top surface of the first alignment member 740 towards a bottom surface of the osteotomy guide 700. The second extension 750 also includes a second channel 752 extending into the second extension 750 from a top surface of the first alignment member 740 towards the bottom surface of the osteotomy guide 700. The channels 748, 752 may be, for example, configured or sized and shaped to receive and retain a temporary fixator, such as, a k-wire. As the first alignment member 740 is angled relative to a top surface of the body 710, the extensions 746, 750 are also angled as the extensions 746, 750 extend from the first side to the second side of the first alignment member 740. Referring now to FIGS. 76, 78, 81 and 82, a first bone contacting portion 754 of the first alignment member 740 is shown. The first bone contacting portion 754 includes a first arm 756 extending away from a first side of the first alignment member 740 and a second arm 758 extending away from a second side of the first alignment member 740. The first and second arms 756, 758 are, for example, configured or sized and shaped to engage a portion of the tibia, as shown in FIGS. 73 and 74.

Referring now to FIGS. 73-82, the second alignment member 760 is shown. The second alignment member 760 includes a first protrusion 762 and a second protrusion 764 each extending away from a first side of the second alignment member 760. The first protrusion 762 is positioned between the first end and a midpoint of the second alignment member 760, while the second protrusion 764 is positioned between the midpoint and a second end of the second alignment member 760. The first protrusion 762 includes at least one relief 763 extending into the first protrusion 762. The second protrusion 764 also includes at least one relief 765 extending into the second protrusion 764. A first alignment opening 766 extends through the first protrusion 762 to the second side of the second alignment member 760. A second alignment opening 768 extends through the second protrusion 764 to the second side of the second alignment member 760. The alignment openings 766, 768 may be, for example, configured or sized and shaped to receive a temporary fixator, such as, a k-wire or the like.

As shown in FIGS. 76, 78, 81 and 82, the second alignment member 760 also includes a second bone contacting portion 770 positioned on the second side of the second alignment member 760. The second bone contacting portion 770 includes a first arm 772 and a second arm 774 extending away from the first side of the second alignment member 760. The first arm 772 is positioned superior to and spaced apart from the second arm 774. The first arm 772 may have a width and length smaller than the width and length of the second arm 774. The second bone contacting portion 770 may also include a third arm 776 extending away from the second side of the second alignment member 760. The third arm 776 may have, for example, a length shorter than both the first and second arms 772, 774. The second bone contacting portion 770 may also include a recessed region 778 extending into the second alignment member 760 from the second side. The recessed region 778 may include a first projection 780 surrounding the first alignment opening 766 and a second projection 782 surrounding the second alignment opening 768. The projections 766, 768 may extend away from the surface of the recessed region 778.

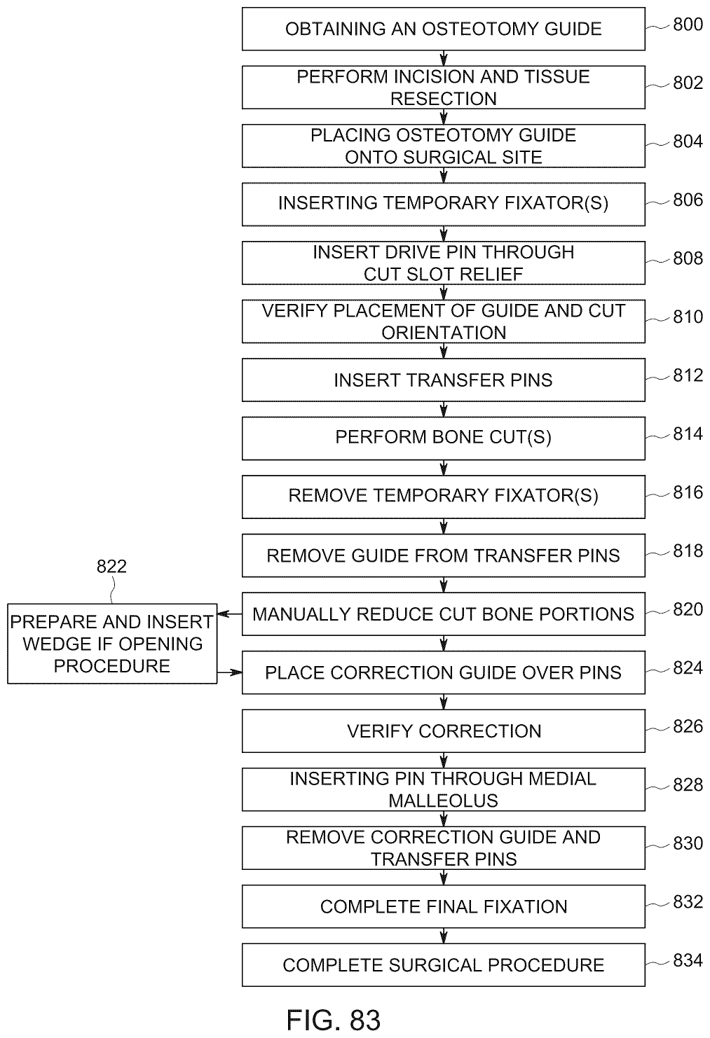

Referring now to FIG. 83, a method for using the osteotomy guide 700 is shown. In some aspects, one or more of the components described herein and incorporated in the method of FIG. 83 may be patient specific. For example, one or more of the components (e.g., the osteotomy guide 700, etc.) may be generated (e.g., manufactured) with one or more features specific to an anatomy of the patient based on patient data. Further to the previous example, the alignment guide 700 may be generated to include a surface geometry matched (e.g., complimentary) to a surface of a bone of a patient to facilitate placement of the osteotomy guide 700 relative to the bone of the patient and thus guide a physician in performing various steps of the method of FIG. 83 with respect to the anatomy of a specific patient. The method includes obtaining an osteotomy guide 800. After the guide is selected, an incision is performed and tissue resected 802. A surgeon may optionally fit the guide onto a bone model of the patient to see the expected fit. Next, the guide is placed onto the patient in the surgical site 804 and at least one temporary fixator is inserted into the pin holes 806. The temporary fixator may be, for example, an olive wire. Next, a drive pin may be inserted through the cut slot relief 808. Alternatively, a sawblade may be placed into the slot. Then, fluoroscopy may be taken to verify the guide placement and cut orientation 810. If the drive pin was used, then the drive pin provides a location for lateral plating of the tibia. If desired, a lateral plate may be attached to the tibia to act as an additional hinge and preserve lateral translation of the tibia. After the placement is verified, the transfer pins may be placed into the proximal channels and distal alignment openings 812. If desired, the transfer pins may then be trimmed to allow for better access to the osteotomy cut slot. Once the transfer pins are in place, the osteotomy may be performed by inserting a saw blade through the slot and cutting the bone 814. Next, the temporary fixation may be removed 816 and the guide may be removed from the transfer pins 818. The proximal transfer pins may be, for example, popped out of the channels of the converging pin trajectories to allow for the guide to be slid off the distal transfer pins. Then, the cut bone portions may be manually reduced 820. If an opening procedure is being performed and a wedge used, the wedge may then be prepared and inserted into the opening between the two bone portions 822. A correction guide may then be placed over the pins based on the planned correction position 824. The correction may then be verified 826 by, for example, the post-op model and/or fluoroscopy. A pin may then be inserted through the medial malleolus to temporarily fix the correction in place 828. A correction guide may optionally be used for insertion of the pin to fix the correction. In addition, a secondary pin may optionally be used to assist with temporary fixation of the correction. Additional verification with fluoroscopy may also optionally be performed. Once the desired correction is achieved, the correction guide and transfer pins may be removed from the surgical site 830. Next, final fixation may be completed 832 by, for example, using a plate to secure the bone portions in the corrected orientation. Finally, the surgical procedure is completed and the patient is closed 834.

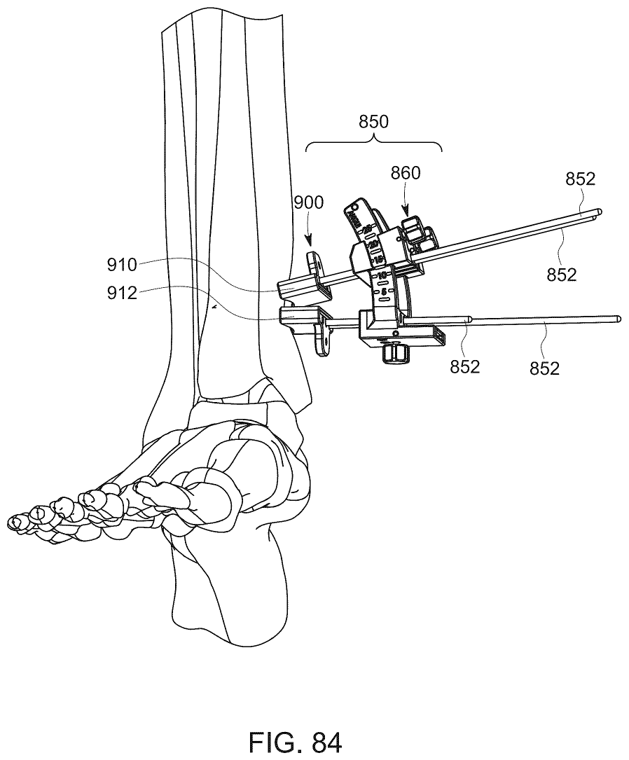

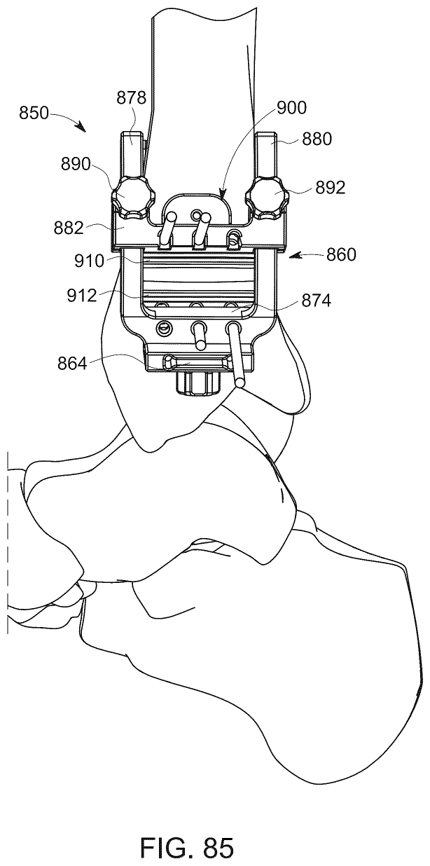

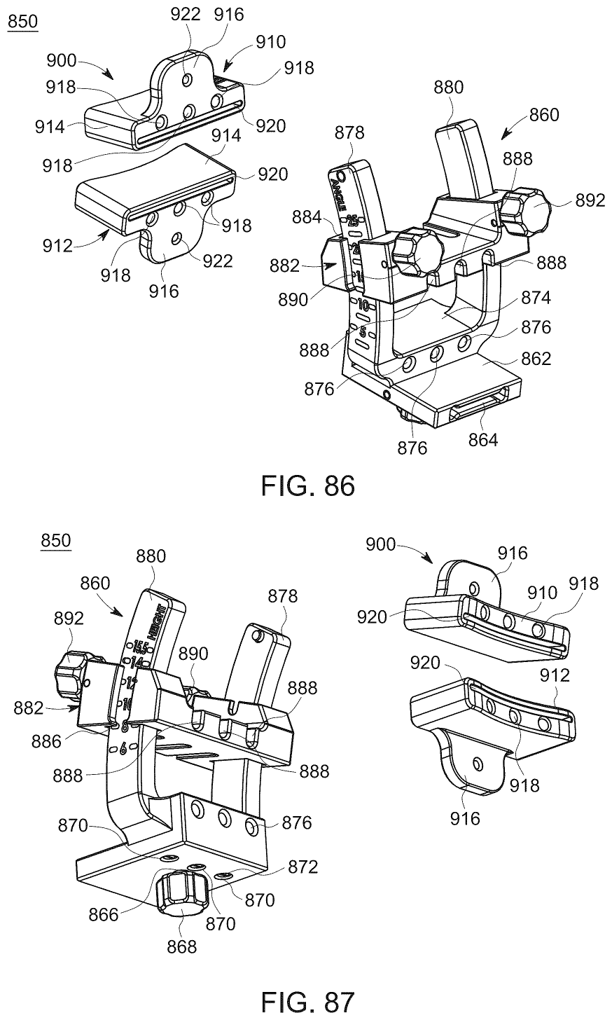

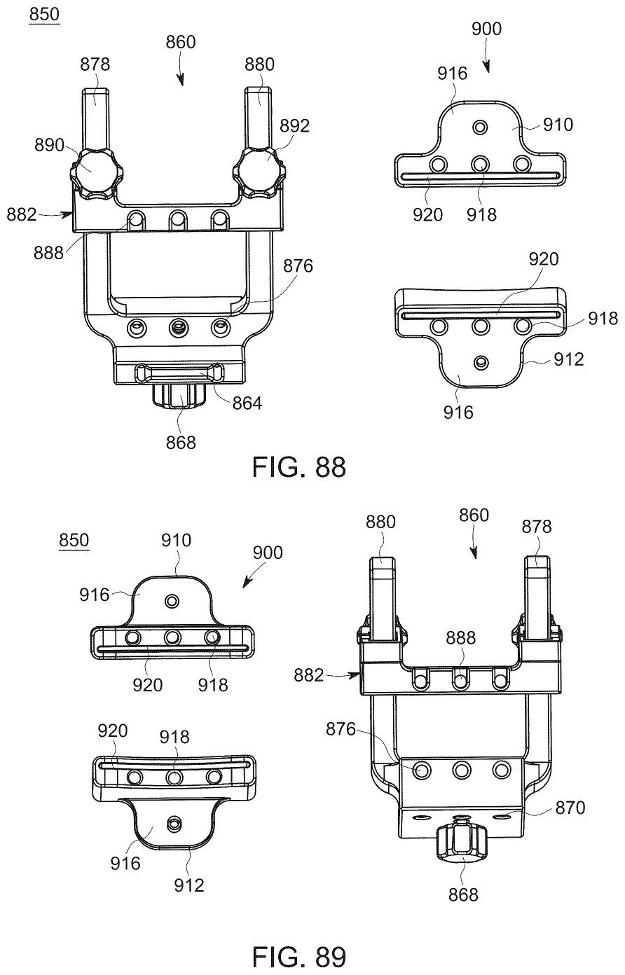

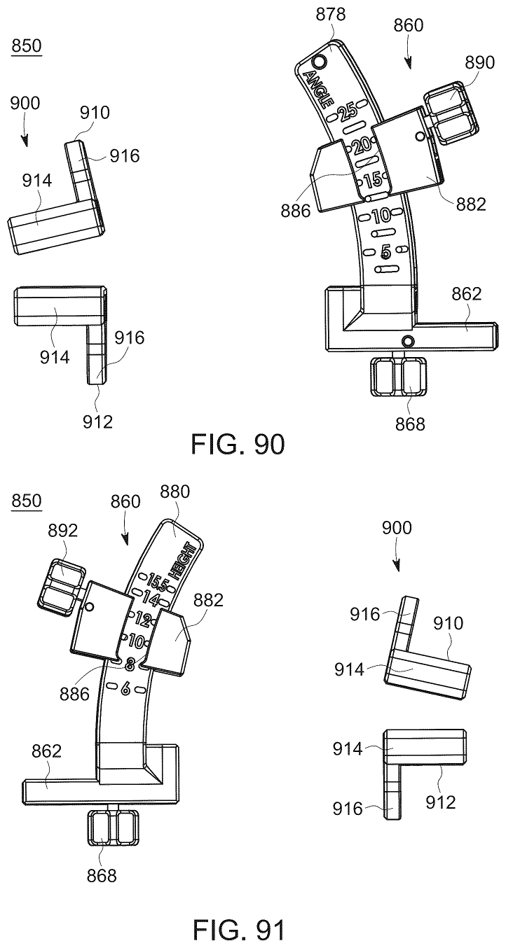

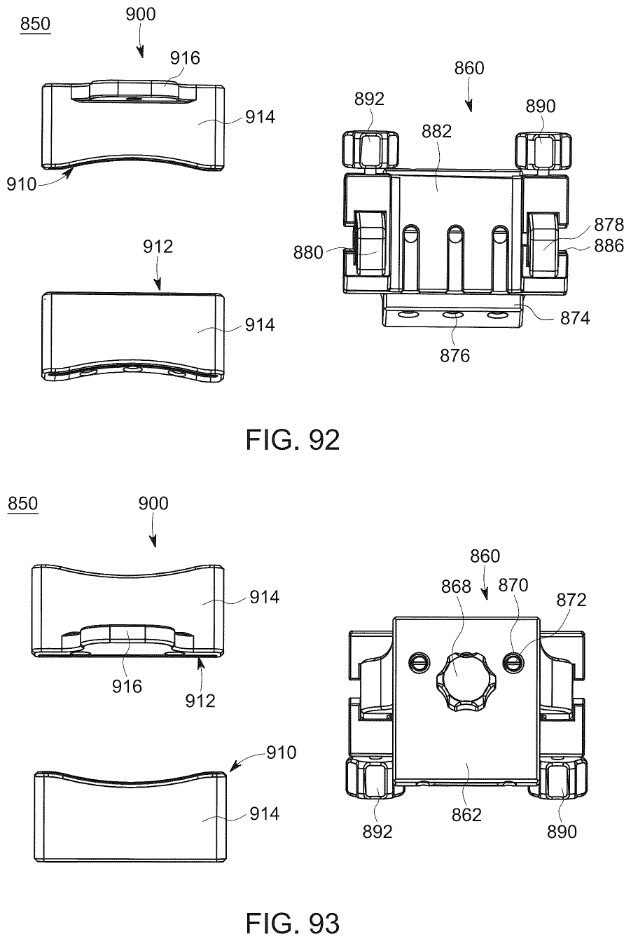

Referring now to FIGS. 84-93, an osteotomy system 850 is shown. The osteotomy system 850 includes an osteotomy jig 860 and a cut guide system 900. The jig 860 includes a base 862 with a slot 864 extending into a portion of the base 862 from a first side of the jig 860 towards a second side. The base 862 also includes a securement opening 866 extending into the base 862 from a bottom surface. The opening 866 may be, for example threaded to receive a first set screw 868 to secure the jig 860 in the desired position along temporary fixation pins 852. The bottom surface of the base 862 may also include at least one opening 870 for receiving a spring plunger 872. As shown in FIG. 87, the at least one opening 870 is three openings spaced along the bottom surface of the base 862. Each opening 870 includes a spring plunger 872. The jig 860 may also include a projection 874 extending away from a portion of the top surface of the base 862. The projection 874 may includes at least one through hole 876 extending through the projection 874 from a first side to a second side. As shown, the at least one through hole 876 may be three through holes 876. The openings 870 may extend from the bottom surface and into the through holes 876 to allow the spring plungers 872 to engage a temporary fixator inserted through the through holes 876. The jig 860 may also include a first arm 878 and a second arm 880. The first arm 878 may extend away from the protrusion 874 on a first end and the second arm 880 may extend away from the protrusion 874 on a second end. The arms 878, 880 may be, for example, curved or arced as the arms 878, 880 extend from the protrusion 874. The first arm 878 may include angle markings, while the second arm 880 may include height markings for the osteotomy procedure. The jig 860 may also include a translating member 882 that moveably couples to the first and second arms 878, 880. The translating member 882 may include a first opening 884 on a first end for receiving the first arm 878 and a second opening 886 on a second end for receiving the second arm 880. The openings 884, 886 may include channels to allow for visualization of the markings on the arms 878, 880. The translating member 882 may also include at least one pathway 888 extending through the translating member 882 from a first side to a second side. As shown, the at least one pathway 888 is three pathways 888. The pathways 888 may, for example, be open on a portion of a top surface of the translating member 882 and a portion of the bottom surface of the translating member 882. The jig 860 may also include a second set screw 890 extending into a first opening in the translating member 882 to engage the first arm 878 to secure the translating member 882 in a desired position. The jig 860 may further include a third set screw 892 extending into a second opening in the translating member 882 to engage the second arm 880 to secure the translating member 882 in a desired position.

With continued reference to FIGS. 84-93, the cut guide system 900 is also shown. The cut guide system 900 may be used once the k-wires are placed into a patient's bones using the jig 860. The cut guide system 900 may include a first cut guide 910 and a second cut guide 912. Each cut guide 910, 912 includes a base 914 and an extension 916 extending away from the exterior surface of each cut guide 910, 912. The base 912 includes at least one alignment opening 918 extending through the base 912 from a first side to a second side. As shown in FIGS. 84-93, the at least one alignment opening 918 is three alignment openings 918 spaced along the base 914 between a first end and a second end. Each base 912 also includes a cut slot 920 extending through the base 912 from a first side to a second side and between a position near the first end and a position near the second end. The extension 916 also includes an opening 922 extending through the extension 916 from a first side to a second side. The first cut guide 910 is a mirror image of the second cut guide 912. For example, the slot 920 of the first cut guide 910 is positioned near a bottom surface and the slot 920 of the second cut guide 910 is positioned near a top surface. In addition, the interior surface of each cut guide 910, 912 may be, for example, curved or arced between the first end and the second end.

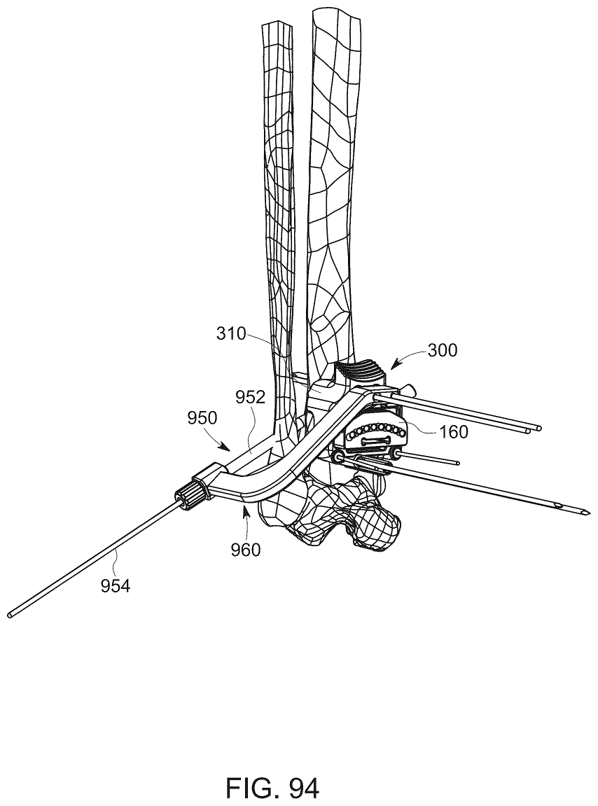

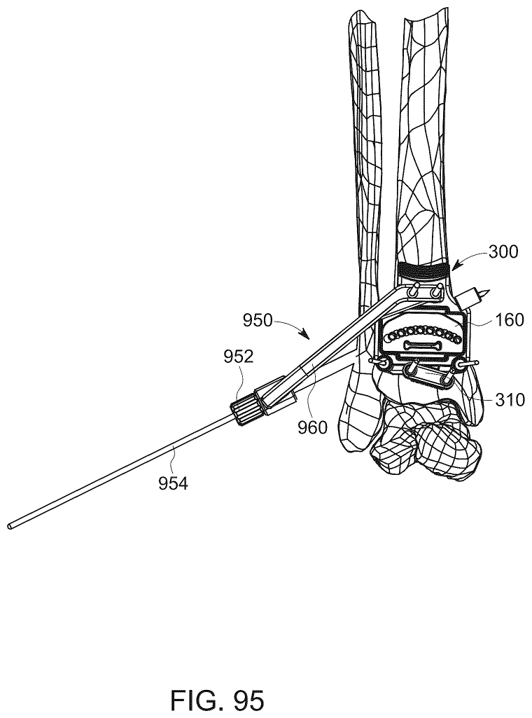

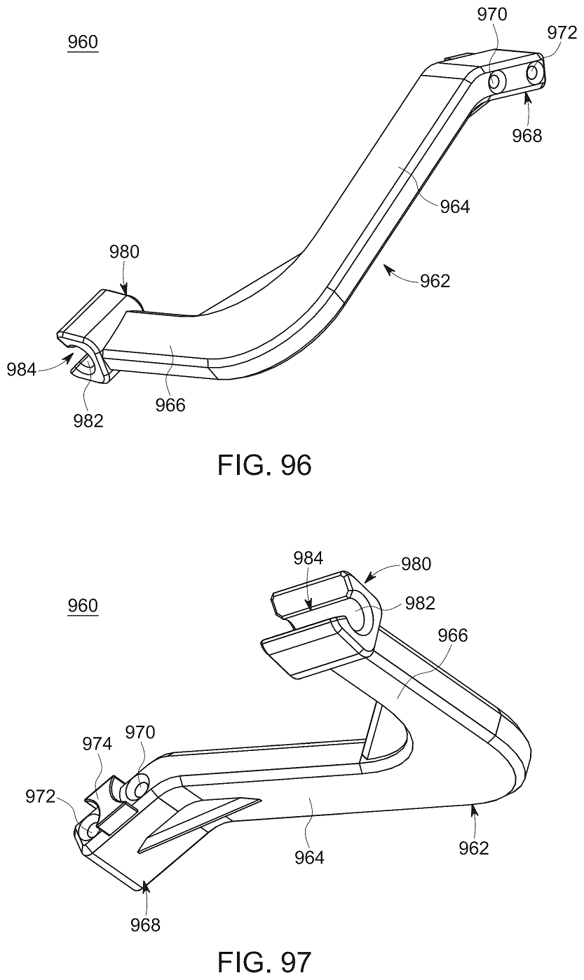

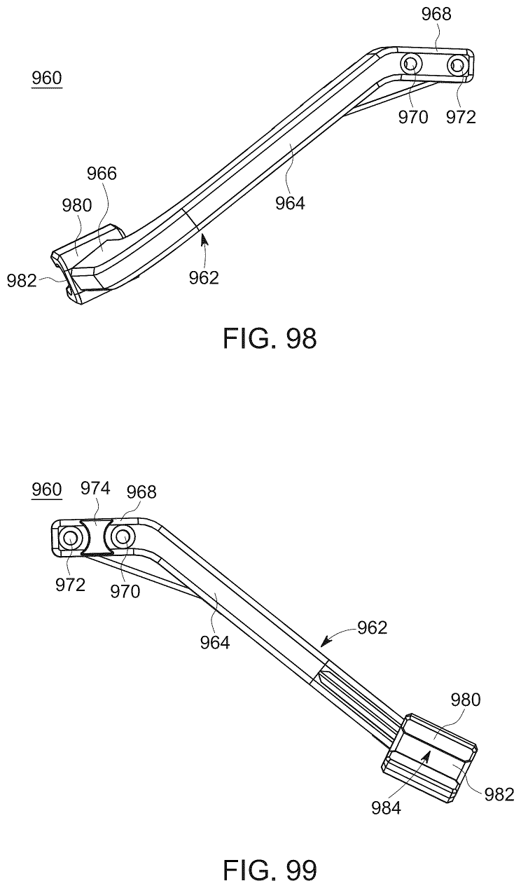

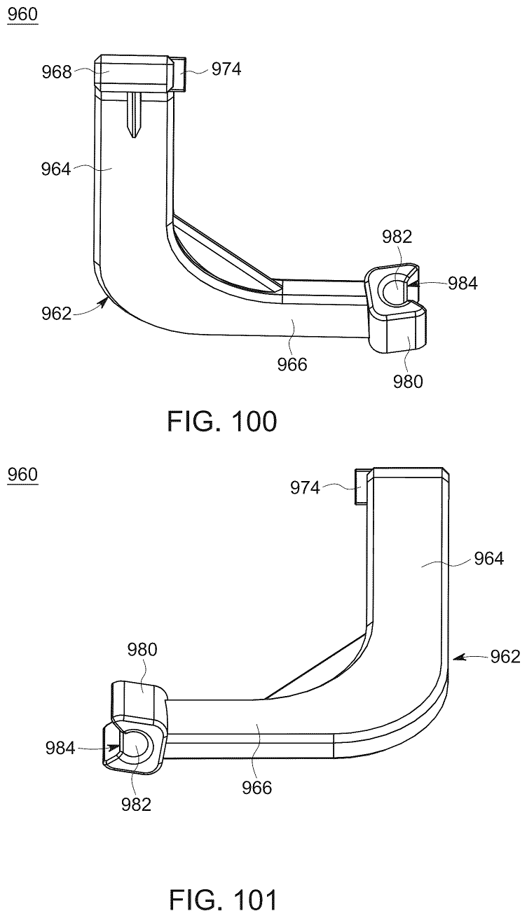

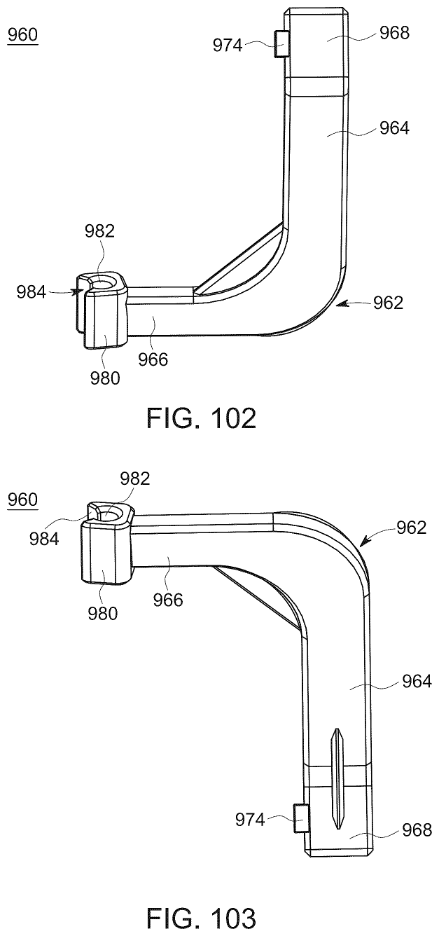

Referring now to FIGS. 94-103, a fibular guide system 950 is shown. The guide system 950 includes a fibular guide 960, a slide pin tube 952, and a fixation pin 954. The guide 960 includes a base 962 with a first portion 964 and a second portion 966. The first portion 964 may be positioned at or about, for example, a 90° angle from the second portion 966. The first portion 964 may also include an alignment portion 968 extending away from an end of the first portion 964 at an angle relative to the first portion 964. The alignment portion 968 may include a first alignment opening 970 and a second alignment opening 972. The alignment openings 970, 972 may extend through the alignment portion 968 from a first side to a second side. The alignment openings 970, 972 may also be spaced apart along the alignment portion 968. The openings 970, 972 may be, for example, configured or sized and shaped to receive a temporary fixator, such as a k-wire. The alignment portion 968 may also include a protrusion 974 extending away from the second side of the alignment portion 968 and positioned between the openings 970, 972. The protrusion 974 may be, for example, shaped to engage the alignment block of an osteotomy guide, for example, alignment block 110. The second portion 966 of the guide 960 may include a coupling member 980 extending away from an end of the second portion 966. The coupling member 980 may include an opening 982 extending through the coupling member 980 from a first end to a second end. The coupling member 980 may also include a channel 984 extending into the opening 982 from an exterior surface of the coupling member 980. The opening 982 may be, for example, configured or sized and shaped to receive the slide pin tube 952. The slide pin tube 952 may be configured or sized and shaped to receive the fixation pin 952 for insertion into a patient's bone, for example, fibula.

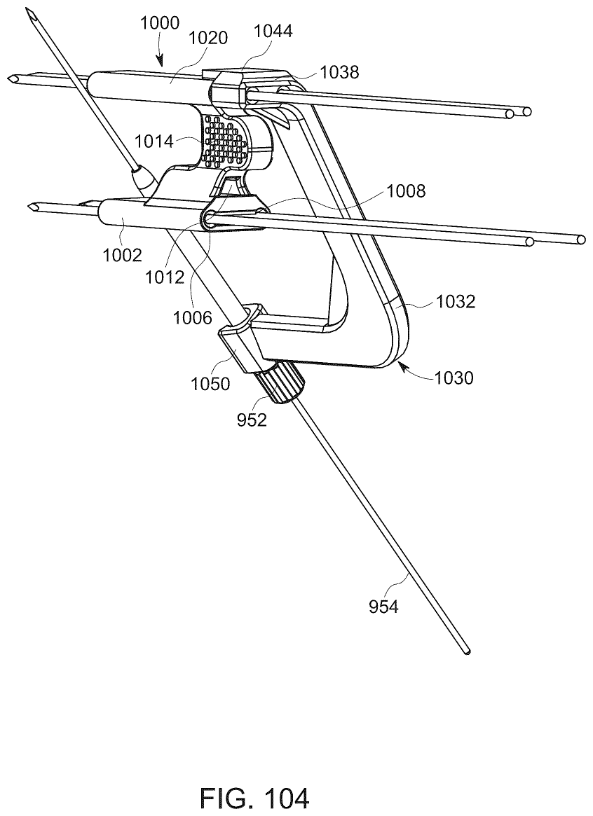

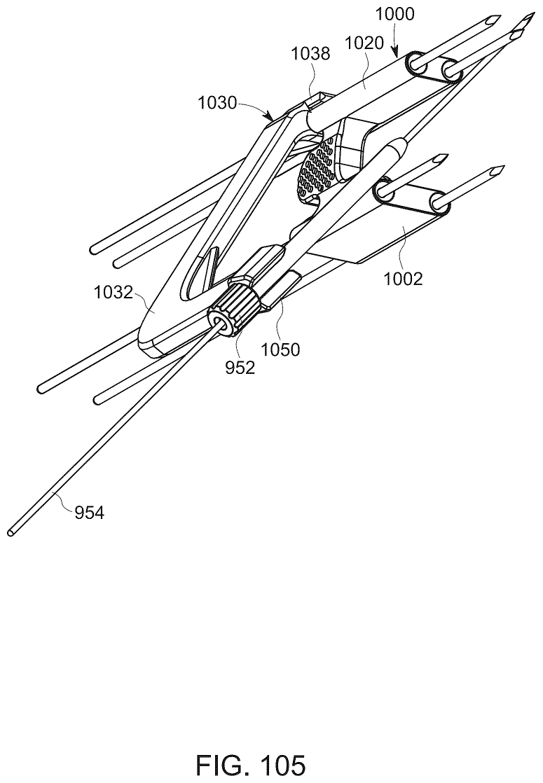

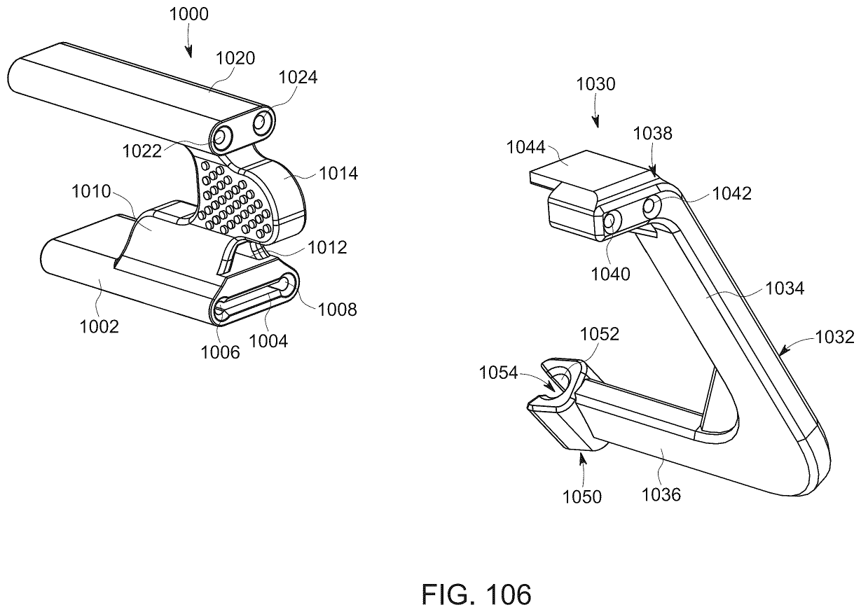

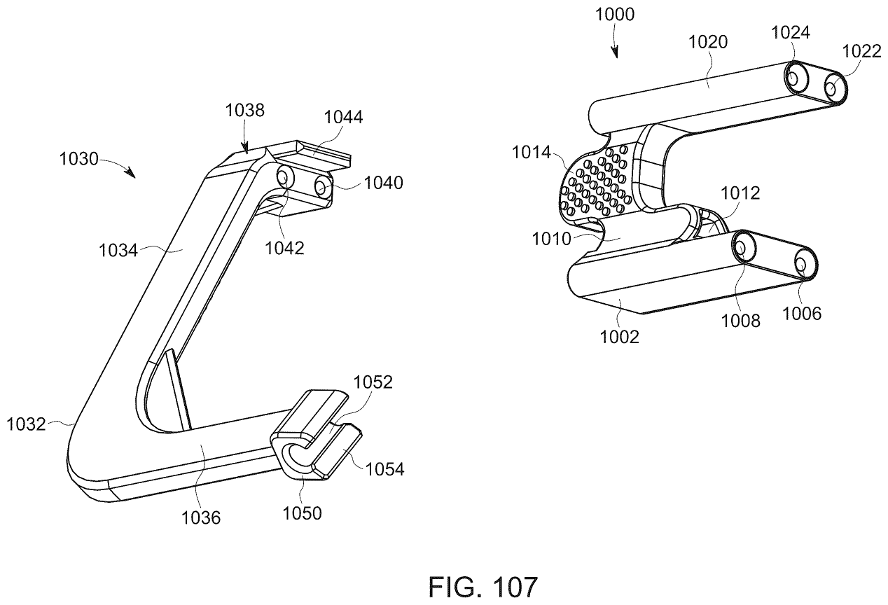

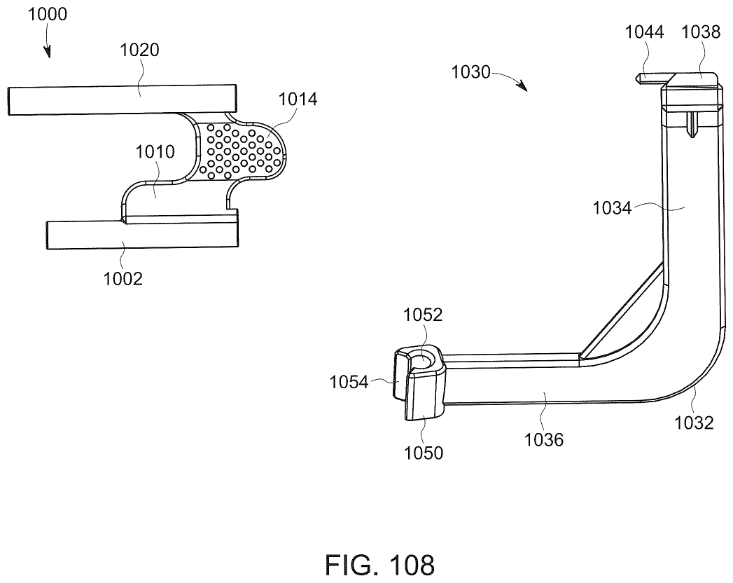

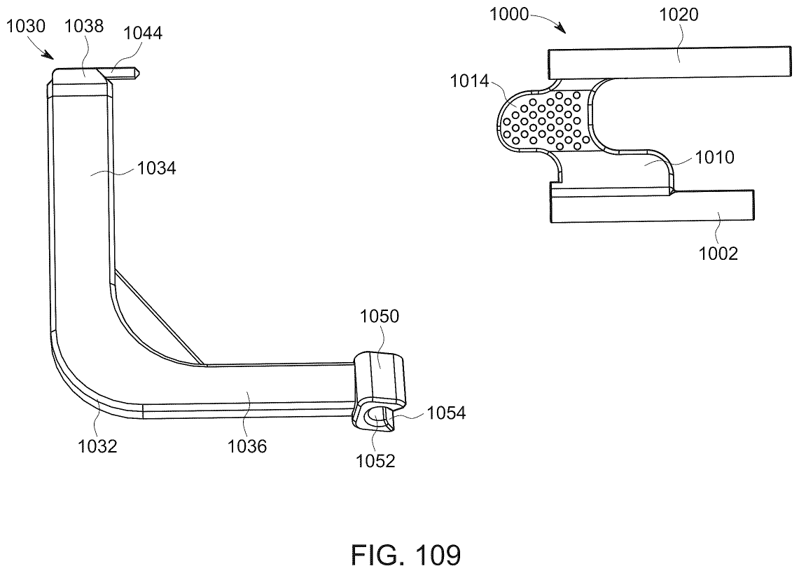

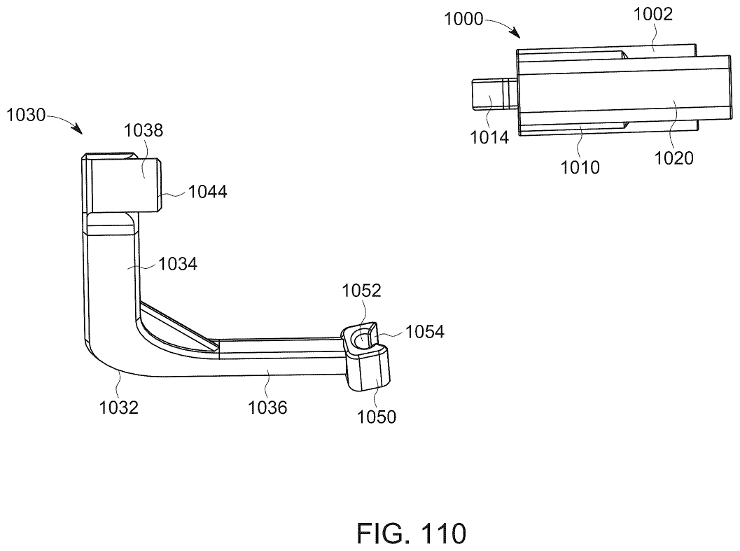

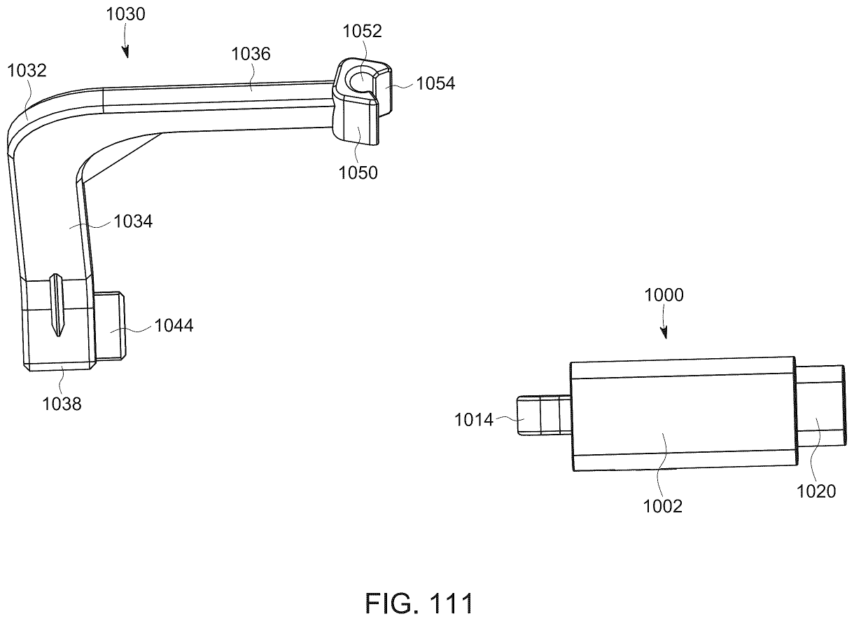

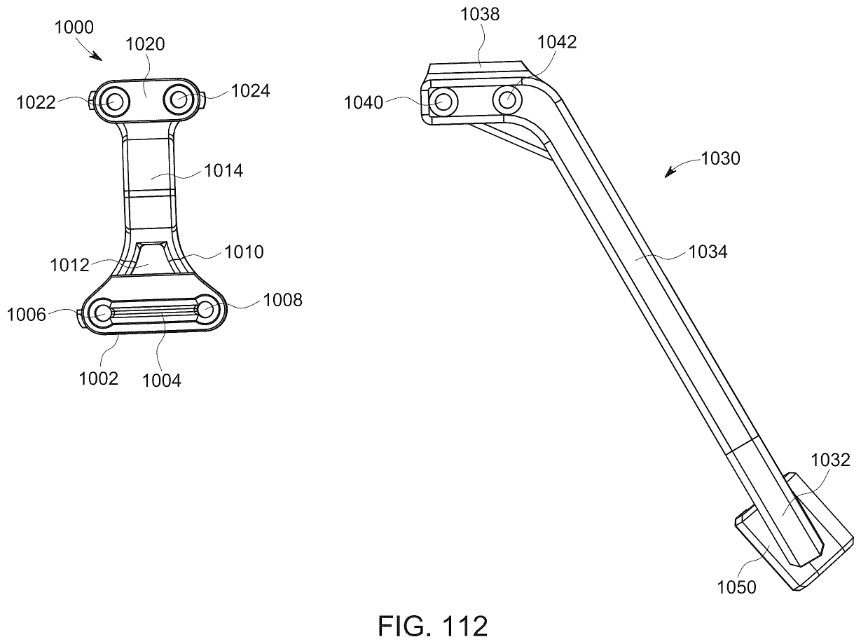

Referring now to FIGS. 104-113, a correction guide 1000 and a tibia screw guide 1030 are shown. The correction guide 1000 includes a base 1002, a protrusion 1010, a handle portion 1014, and an alignment member 1020. The base 1002 includes a slot 1004 extending into a portion of the base 1002 from a first side. The base 1002 also includes a first alignment opening 1006 and a second alignment opening 1008 extending through the base 1002 from the first side to a second side. The first opening 1006 is positioned near a first end of the base 1002 and overlap a portion of the slot 1004. The second opening 1008 is positioned near a second end of the base 1002 and overlaps a portion of the slot 1004. The openings 1006, 1008 are configured or sized and shaped to receive temporary fixators, such as, a pin, k-wire or the like. The correction guide 1000 also includes a protrusion 1010 extending away from a superior or top surface of the base 1002. The protrusion 1010 extends along a portion of the base 1002 between the first side and the second side. The protrusion 1010 also includes a through hole 1012 extending through the protrusion 1010 from the first side to the second side. The correction guide 1000 also includes a handle portion 1014 extending away from a top or superior portion of the protrusion 1010. The handle portion 1014 may extend along a portion of the protrusion 1010. In addition, the handle portion 1014 may extend away from the first side of the protrusion 1010 such that the handle portion 1014 extends beyond the anterior surface of the protrusion 1010. The correction guide 1000 further includes an alignment member 1020 coupled to and extending away from a top surface of the handle portion 1014. The alignment member 1020 is coupled to a second side of the handle portion 1014. The alignment member 1020 includes a first alignment opening 1022 and a second alignment opening 1024. The first alignment opening 1022 may extend through the alignment member 1020 from a first side to a second side and be positioned near a first end of the alignment member 1020. The second alignment opening 1024 may extend through the alignment member 1020 from a first side to a second side and be positioned near a second end of the alignment portion 1020.

With continued reference to FIGS. 104-113, the tibia screw guide 1030 includes a base 1032 with a first portion 1034 and a second portion 1036. The first portion 1034 may be positioned at or about, for example, a 90° angle from the second portion 1036. The first portion 1034 may also include an alignment portion 1038 extending away from an end of the first portion 1034 at an angle relative to the first portion 1034. The alignment portion 1038 may include a first alignment opening 1040 and a second alignment opening 1042. The alignment openings 1040, 1042 may extend through the alignment portion 1038 from a first side to a second side. The alignment openings 1040, 1042 may also be spaced apart along the alignment portion 1038. The openings 1040, 1042 may be, for example, configured or sized and shaped to receive a temporary fixator, such as a k-wire. The alignment portion 1038 may also include a protrusion 1044 extending away from the second side of the alignment portion 1038 at a top of the alignment portion 1038. The protrusion 1044 may be, for example, shaped to engage the correction guide 1000. The second portion 1036 of the guide 1030 may include a coupling member 1050 extending away from an end of the second portion 1036. The coupling member 1050 may include an opening 1052 extending through the coupling member 1050 from a first end to a second end. The coupling member 1050 may also include a channel 1054 extending into the opening 1052 from an exterior surface of the coupling member 1050. The opening 1052 may be, for example, configured or sized and shaped to receive the slide pin tube 952. The slide pin tube 952 may be configured or sized and shaped to receive the fixation pin 952 for insertion into a patient's bone, for example, tibia.

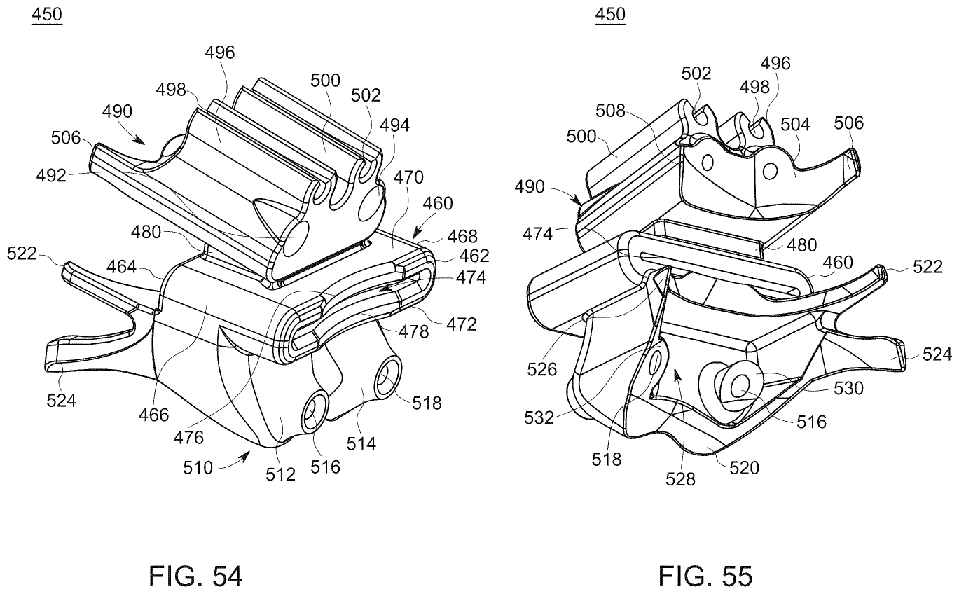

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants may include more or fewer components or features than the embodiments as described and illustrated herein. For example, the components and features of FIGS. 1-16 and FIGS. 18-35 may be used interchangeably and in alternative combinations as would be modified or altered by one of skill in the art. Specifically, osteotomy guides 100, 300 may be used in alternative combinations as would be modified or altered by one of skill in the art. In addition, the components and features of FIGS. 54-61, FIGS. 64-71, and FIGS. 75-82 may be used interchangeably and in alternative combinations as would be modified or altered by one of skill in the art. Specifically, osteotomy guides 450, 550, 700 may be used in alternative combinations as would be modified or altered by one of skill in the art. Accordingly, this detailed description of the currently-preferred embodiments is to be taken in an illustrative, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. A guide system, comprising:
an alignment block; and
a cut guide removably coupled to the alignment block.

2. The guide system of claim 1, wherein the alignment block comprises:
a housing;
a first alignment member extending away from a superior side of the housing; and
a second alignment member extending away from an inferior side of the housing.

3. The guide system of claim 2, wherein the housing comprises:
a through hole extending through the housing from a first side to a second side of the housing; and
at least one recess extending into at least a portion of the through hole.

4. The guide system of claim 3, wherein the at least one recess comprises:
a first recess extending into a portion of a top surface of the through hole; and
a second recess extending into a portion of a bottom surface of the through hole.

5. The guide system of claim 2, wherein the first alignment member comprises:
at least one alignment opening extending through the first alignment member from a first side to a second side; and
wherein the second alignment member comprises:
at least one alignment opening extending through the second alignment member from a first side to a second side.

6. The guide system of claim 5, wherein the guide system further comprises:
a set screw rotatably coupled to the alignment block; and
at least one temporary fixation pin removably insertable into the at least one alignment opening of the first alignment member.

7. The guide system of claim 6, wherein the first alignment member further comprises:
a screw hole for rotatably receiving the set screw, wherein the screw hole is positioned superior to the at least one alignment opening. 8 The guide system of claim 7, wherein the guide system further comprises:
a fastener removably coupled to a second end of the set screw.

9. The guide system of claim 5, wherein the at least one alignment opening of the first alignment member comprises:
a first alignment opening extending in a first direction as the first alignment opening extends through the first alignment member; and
a second alignment opening spaced apart from the first alignment opening and extending in a second direction as the second alignment opening extends through the second alignment opening; and
wherein the first direction converges with the second direction as the first and second alignment openings extend from a first side to a second side of the first alignment member
wherein the at least one alignment opening of the second alignment member comprises:
a first alignment opening; and
a second alignment opening spaced apart from the first alignment opening.

10. The guide system of claim 3, wherein the housing further comprises:
a first opening extending through a first end of the housing, wherein the first opening receives a first fastener removably coupled to the cut guide;
a second opening extending through a second end of the housing, wherein the second opening receives a second fastener removably coupled to the cut guide; and
at least one first pin hole extending through the housing from a first side to a second side.

11. The guide system of claim 10, wherein the cut guide comprises:
at least one set of drill openings extending through the cut guide from a first side to a second side; and
at least one protrusion extending away from at least one of a superior surface and an inferior surface of the cut guide.

12. The guide system of claim 11, wherein the at least one set of drill openings comprises:
a first set of drill openings; and
a second set of drill openings offset from the first set of drill openings; and
wherein the at least one protrusion comprises:
a first protrusion extending away from the superior surface of the cut guide; and
a second protrusion extending away from the inferior surface of the cut guide.

13. The guide system of claim 11, wherein the cut guide further comprises:
a first screw hole extending into a first end of the cut guide, wherein the first screw hole receives the first fastener;
a second screw hole extending into a second end of the cut guide, wherein the second screw hole receives the second fastener; and
a slot extending through the cut guide from a first side to a second side of the cut guide.

14. The guide system of claim 1, further comprising:
a fibular guide system removably coupled to the alignment block, wherein the fibular guide system comprises:
a base;
a first portion positioned at a first end of the base, wherein the first portion comprises:
an alignment portion extending away from a first end of the first portion; and
a second portion positioned at a second end of the base, wherein the first portion is positioned at a 90° angle from the second portion.

15. The guide system of claim 14, wherein the alignment portion comprises:
a first alignment opening extending through the alignment portion;
a second alignment opening extending through the alignment portion, and wherein the first alignment opening is spaced apart from the second alignment opening along a length of the alignment portion; and
a protrusion extending away from a second side of the alignment portion between the first alignment opening and the second alignment opening, wherein the protrusion engages the alignment block.

16. The guide system of claim 14, wherein the second portion comprises:
a coupling member extending away from a second end of the second portion, wherein the coupling member comprises:
an opening extending through the coupling member from a first end to a second end; and
a channel extending through an exterior surface of the coupling member and into the opening.

17. The guide system of claim 14, further comprising:
a slide pin tube removably inserted through an opening in a coupling member of a second portion of the fibular guide system; and
a fixation pin removably inserted through the slide pin tube.

18. A surgical method of using an osteotomy guide, wherein the method comprises:
obtaining the osteotomy guide, wherein the osteotomy guide comprises:
an alignment block; and
a cut guide removably coupled to the alignment block;
positioning the alignment block on an anterior portion of ankle bones;
inserting the cut guide into the alignment block; and
drilling through the cut guide and the ankle bones.

19. The surgical method of using an osteotomy guide of claim 18, further comprising:
obtaining a fibular guide system;
coupling the fibular guide system to the alignment block; and
inserting a k-wire through the fibular guide system to secure a fibula of the ankle bones.

20. The surgical method of using an osteotomy guide of claim 19, wherein the alignment block comprises:
a body;
a first alignment member coupled to and extending away from a superior surface of the body; and
a second alignment member coupled to and extending away from an inferior surface of the body; and
wherein the method further comprises:
inserting at least one k-wire through the first alignment member and into the ankle bones; and
inserting at least one k-wire through the second alignment member and into the ankle bones.
